SEQUENCE LISTING (35 sequences)

SEQ ID NO: 1 - protein, 271 aa, Artificial Sequence
  amino acid sequence of single-chain (scFv) antibody against platelet marker CD41
MAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLRQPKAAPSVTLFPPSS

SEQ ID NO: 2 - protein, 127 aa, Artificial Sequence
  heavy chain VH amino acid sequence of scFv to CD41
MAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTV

SEQ ID NO: 3 - protein, 140 aa, Artificial Sequence
  light chain VL amino acid sequence of scFv to CD41
ASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLRQPKAAPSVTLFPPSS

SEQ ID NO: 4 - protein, 439 aa, Artificial Sequence
  amino acid sequence of extracellular domain of CD39
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 5 - DNA, 2199 bp, Artificial Sequence
  nucleotide sequence of scFv-CD39
atggcggaggtgcagctggtggagtctgggggaggcttggtacagcctggggggtccctgagactctcctgtgcagcctctggattcatgtttagcaggtatgccatgagctgggtccgccaggctccagggaaggggccagagtgggtctcaggtattagtggtagtggtggtagtacatactacgcagactccgtgaagggccggttcaccgtctccagagacaattccaagaacacgctgtatctgcaaatgaacagcctgagagccgaggacactgccgtgtattactgtgcgagaatttttacgcatcggtcgcgtggtgacgtcccggaccaaactagctttgactactggggccagggaaccctggtcaccgtctcctcagggagtgcatccgccccaaagcttgaagaaggtgaattttcagaagcacgcgtaagctctgaactgactcaggaccctgctgtgtctgtggccttgggacagacagtcaggatcacatgccaaggagacagcctcagaaacttttatgcaagctggtaccagcagaagccaggacaggcccctactcttgtcatctatggtttaagtaaaaggccctcagggatcccagaccgattctctgcctccagctcaggaaacacagcttccttgaccatcactggggctcaggcggaagatgaggctgattattactgcctgctctactatggtggtggtcagcagggagtgttcggcggagggaccaagctgaccgtcctacgtcagcccaaggctgccccctcggtcactctgttcccgccctcttctgcggccgcaacccagaacaaagcattgccagaaaacgttaagtatgggattgtgtcttctgcggccgcaacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacc

SEQ ID NO: 6 - protein, 713 aa, Artificial Sequence
  amino acid sequence of scFv-CD39
MAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLRQPKAAPSVTLFPPSSAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 7 - protein, 279 aa, Artificial Sequence
  amino acid sequence of scFVmut
AEVQLVQSGAEVNKPGASVKVSCKASGYTFTGYYMHWVRQAPGQGLEWMGWINPNSGGTNYAQKFQGWVTMTRDTSISTAYMELSRLRSDDTAVYYCARGRALYNANDRSPNWFDPWGQGTLVTVSSGSASAPTLKLEEGEFSEARVQAVLTQPPSVSVAPGQTARITCGGNNIGSKSVQWYQQKPGQAPVLVVYDDSDRPSGIPERFSGSNSGNMATLTISRVEAGDEADYYCQVWDSSSDHVVFGGGTKLTVLGGKPIPNPLLGLDSTLPETGGLEE

SEQ ID NO: 8 - protein, 510 aa, Artificial Sequence
  amino acid sequence of human CD39
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 9 - protein, 125 aa, Artificial Sequence
  heavy chain VH amino acid sequence of scFvmut to CD41
AEVQLVQSGAEVNKPGASVKVSCKASGYTFTGYYMHWVRQAPGQGLEWMGWINPNSGGTNYAQKFQGWVTMTRDTSISTAYMELSRLRSDDTAVYYCARGRALYNANDRSPNWFDPWGQGTLVTV

SEQ ID NO: 10 - protein, 150 aa, Artificial Sequence
  light chain VL amino acid sequence of scFvmut to CD41
ASAPTLKLEEGEFSEARVQAVLTQPPSVSVAPGQTARITCGGNNIGSKSVQWYQQKPGQAPVLVVYDDSDRPSGIPERFSGSNSGNMATLTISRVEAGDEADYYCQVWDSSSDHVVFGGGTKLTVLGGKPIPNPLLGLDSTLPETGGLEE

SEQ ID NO: 11 - protein, 775 aa, Artificial Sequence
  amino acid sequence of scFVmut-CD39
HHHHHHHHRRAVRSLVPSSDPLQCGGILAEVQLVQSGAEVNKPGASVKVSCKASGYTFTGYYMHWVRQAPGQGLEWMGWINPNSGGTNYAQKFQGWVTMTRDTSISTAYMELSRLRSDDTAVYYCARGRALYNANDRSPNWFDPWGQGTLVTVSSGSASAPTLKLEEGEFSEARVQAVLTQPPSVSVAPGQTARITCGGNNIGSKSVQWYQQKPGQAPVLVVYDDSDRPSGIPERFSGSNSGNMATLTISRVEAGDEADYYCQVWDSSSDHVVFGGGTKLTVLGGKPIPNPLLGLDSTLPETGGLEEAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTTRGGPEQKLISEEDLNSAVDHHHHHH

SEQ ID NO: 12 - protein, 1039 aa, Artificial Sequence
  amino acid sequence of human GPIIb
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

SEQ ID NO: 13 - protein, 788 aa, Artificial Sequence
  amino acid sequence of human GPIIIa
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 14 - DNA, 2328 bp, Artificial Sequence
  nucleotide sequence of scFv-CD39mut
caccatcatcaccatcaccatcataggcgcgccgtacgaagcttggtaccgagctcggatccactccagtgtggtggaattctcgccgaggtgcagctggtgcagtctggcgccgaagtgaacaagcctggagccagcgtgaaggtgtcctgcaaggccagcggctacaccttcaccggctactacatgcactgggtccgacaggccccaggacagggcctggaatggatgggctggatcaaccccaacagcggcggcaccaactacgcccagaaattccagggctgggtcaccatgacccgggacaccagcatcagcaccgcctacatggaactgagccggctgcggagcgacgacaccgccgtgtactactgcgccagaggcagagccctgtacaacgccaacgacagaagccccaattggttcgacccctggggacagggcaccctggtcacagtgtctagcggcagcgccagcgcccccaccctgaaactggaagagggcgagttcagcgaggccagagtgcaggccgtgctgacccagcctccctccgtgtctgtggctcctggacagaccgcccggatcacatgcggcggcaacaacatcggcagcaagagcgtgcagtggtatcagcagaagcccggccaggcccccgtgctggtggtgtacgacgacagcgaccggcccagcggcatccccgagagattcagcggcagcaacagcggcaacatggccaccctgaccatcagccgggtggaagccggcgacgaggccgactactactgccaggtctgggacagcagcagcgaccacgtggtgttcggcggaggcaccaagctgacagtgctgggcggcaagcccatccccaaccctctgctgggcctggacagcaccctgcctgagacaggcggcctggaagaagcggccgcaacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccaccactcgaggagggcccgaacaaaaactcatctcagaagaggatctgaatagcgccgtcgaccatcatcatcatcatcattga

SEQ ID NO: 15 - protein, 5 aa, Artificial Sequence
  amino acid sequence of scFv VH CDR1
RYAMS

SEQ ID NO: 16 - protein, 17 aa, Artificial Sequence
  amino acid sequence of scFv VH CDR2
GISGSGGSTYYADSVKG

SEQ ID NO: 17 - protein, 21 aa, Artificial Sequence
  amino acid sequence of scFv VH CDR3
CARIFTHRSRGDVPDQTSFDY

SEQ ID NO: 18 - protein, 11 aa, Artificial Sequence
  amino acid sequence of scFv VL CDR1
QGDSLRNFYAS

SEQ ID NO: 19 - protein, 7 aa, Artificial Sequence
  amino acid sequence of scFv VL CDR2
GLSKRPS

SEQ ID NO: 20 - protein, 11 aa, Artificial Sequence
  amino acid sequence of scFv VL CDR3
LLYYGGGQQGV

SEQ ID NO: 21 - protein, 127 aa, Artificial Sequence
  amino acid sequence of a scFv VH
EVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSS

SEQ ID NO: 22 - protein, 107 aa, Artificial Sequence
  amino acid sequence of a scFv VL
SELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVL

SEQ ID NO: 23 - protein, 253 aa, Artificial Sequence
  amino acid sequence of a scFv to CD41
EVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVL

SEQ ID NO: 24 - protein, 711 aa, Artificial Sequence
  amino acid sequence of a scFv-CD39 fusion protein
EVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLRQPKAAPSVTLFPPSSAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 25 - protein, 695 aa, Artificial Sequence
  amino acid sequence of a scFv-CD39 fusion
EVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 26 - protein, 697 aa, Artificial Sequence
  amino acid sequence of a scFv-CD39 fusion
MAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 27 - protein, 796 aa, Artificial Sequence
  amino acid sequence of a scFV-CD39 fusion
METDTLLLWVLLLWVPGSTGDAAQPARRAVRSLVPSSDPLQCGGILHHHHHHHHRRAMAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLRQPKAAPSVTLFPPSSAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTTRGGPEQKLISEEDLNSAVDHHHHHH

SEQ ID NO: 28 - protein, 780 aa, Artificial Sequence
  amino acid sequence of a scFv-CD39 fusion protein
METDTLLLWVLLLWVPGSTGDAAQPARRAVRSLVPSSDPLQCGGILHHHHHHHHRRAMAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTTRGGPEQKLISEEDLNSAVDHHHHHH

SEQ ID NO: 29 - protein, 19 aa, Artificial Sequence
  amino acid sequence of a flexible linker
GSASAPKLEEGEFSEARVS

SEQ ID NO: 30 - protein, 46 aa, Artificial Sequence
  amino acid sequence of a signal peptide
METDTLLLWVLLLWVPGSTGDAAQPARRAVRSLVPSSDPLQCGGIL

SEQ ID NO: 31 - protein, 586 aa, Homo sapiens
AHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGG

SEQ ID NO: 32 - protein, 1363 aa, Artificial Sequence
  amino acid sequence of a scFv-HSA-CD39 fusion
METDTLLLWVLLLWVPGSTGDAAQPARRAMAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLRQPKAAPSVTLFPPSSAISMDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGGGAAATQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTTRGGPEQKLISEEDLNSAVDHHHHHH

SEQ ID NO: 33 - protein, 1280 aa, Artificial Sequence
  amino acid sequence of a scFv-HSA-CD39 fusion
  X (254)..(254): X is any amino acid
  X (254)..(254): X is any amino acid
  X (841)..(841): X is any amino acid
EVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLXAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGXTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 34 - protein, 1319 aa, Artificial Sequence
  amino acid sequence of a scFv-HSA-CD39 fusion
METDTLLLWVLLLWVPGSTGDAAQPARRAMAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGGGSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHST

SEQ ID NO: 35 - protein, 1345 aa, Artificial Sequence
  amino acid sequence of a scFv-HSA-CD39 fusion
METDTLLLWVLLLWVPGSTGDAAQPARRAMAEVQLVESGGGLVQPGGSLRLSCAASGFMFSRYAMSWVRQAPGKGPEWVSGISGSGGSTYYADSVKGRFTVSRDNSKNTLYLQMNSLRAEDTAVYYCARIFTHRSRGDVPDQTSFDYWGQGTLVTVSSGSASAPKLEEGEFSEARVSSELTQDPAVSVALGQTVRITCQGDSLRNFYASWYQQKPGQAPTLVIYGLSKRPSGIPDRFSASSSGNTASLTITGAQAEDEADYYCLLYYGGGQQGVFGGGTKLTVLGGGGSAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGGGSTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTTRGGPEQKLISEEDLNSAVDHHHHHH

(19) United States
(12) Patent Application Publication
(10) Pub. No.: US 2023/0399376 A1
(43) Pub. Date: Dec. 14, 2023

(54) BINDING PROTEINS COMPRISING THE EXTRACELLULAR DOMAIN OF CD39 AND METHODS OF TREATING OR PREVENTING NEUROLOGICAL DISEASES

(71) Applicant: BAKER HEART AND DIABETES INSTITUTE, Melbourne (AU)

(72) Inventors: Karlheinz PETER, Melbourne (AU); Xiaowei WANG, Melbourne (AU); Jacqueline ORIAN, Bundoora (AU)

(21) Appl. No.: 17/614,698

(22) PCT Filed: May 27, 2020
(86) PCT No.: PCT/AU2020/050527

§ 371 Date: Nov. 29, 2021

(30) Foreign Application Priority Data
May 27, 2019 (AU) 2019901808

(51) Int. Cl. C07K 14/705; C07K 16/28; A61P 25/00

(52) U.S. Cl. CPC C07K 14/70596; C07K 16/2842; C07K 2319/31; C07K 2317/622; C07K 2317/565; A61P 25/00

(57) ABSTRACT

The present invention provides a method of treating or preventing an inflammatory neurological disease in a subject, the method comprising administering to the subject a protein comprising an extracellular domain of CD39. The present invention also relates to binding proteins comprising an extracellular domain of CD39.

### CROSS REFERENCE TO RELATED APPLICATIONS

The present disclosure is the national phase under 35 U.S.C 371 of PCT/AU2020/050527 filed on May 27, 2020, which claims priority to Australian Patent Application No. 2019901808, filed with the Australian Patent Office on May 27, 2019, both of which are incorporated herein by reference in their entireties.

### INCORPORATION BY REFERENCE OF SEQUENCE LISTING

A Sequence Listing is provided herewith as a text file entitled “10903-006US1_Replacement_Sequence_Listing.txt” created on May 31, 2023, and having a size of 144,050 bytes. The contents of the text file are incorporated by reference herein in their entirety.

### FIELD

The present disclosure relates to a CD39 binding protein and uses thereof in treating or preventing an inflammatory neurological disease in a subject.

### BACKGROUND

Inflammatory neurological diseases are a class of conditions in which a subject's immune system targets or attacks components of the neurological system. These diseases can result from the immune system attacking, for example, neurons, Schwann cells or other cells of the nervous system myelin or neurotransmitters. In some cases, the inflammatory neurological disease may be a complication or a component of an existing disease. Exemplary inflammatory neurological diseases include multiple sclerosis, systemic lupus erythematosus (SLE), Guillain-Barre syndrome, Lambert-Eaton myasthenic syndrome, myasthenia gravis, transverse myelitis, leukodystrophy or progressive multifocal leukoencephalopathy.

Multiple sclerosis (MS) is a common inflammatory neurological disease. It is a demyelinating disease of the central nervous system, interfering with nerve impulses within the brain, spinal cord and optic nerves. In 2015, over 2 million people were diagnosed worldwide. Most people are diagnosed between the ages of 20-40, and the disease affects approximately three times as many women as men. MS is a heterogeneous disorder based on clinical course, magnetic resonance imaging (MRI) scan assessment, and pathology analysis of biopsy and autopsy material. The disease manifests itself in a large number of possible combinations of deficits, including spinal cord, brainstem, cranial nerve, cerebellar, cerebral, and cognitive syndromes.

There is no known cure for MS. Currently available treatment options include administration of steroidal medication (e.g., methylprednisolone), immune suppressants (e.g., methotrexate or mitozantrone) and immunotherapy regimens. However, these drugs are symptomatic therapies (i.e., reduce specific symptoms) or reduce the risk of relapses and disease progression (i.e., disease modifying therapies). Medications used to treat MS, while modestly effective, can have side effects and be poorly tolerated. Physical therapy can also help with people's ability to function. The long-term outcome is difficult to predict, with life expectancy on average 5 to 10 years lower than that of an unaffected population.

Although platelets have been investigated in the context of hemostasis and thrombosis for decades, their role in inflammation emerged relatively recently. Platelets express a plethora of receptors and can release a wide range of inflammatory mediators that are not associated with their classical roles, but with inflammation, immunity and tissue repair. To date, platelets have been implicated in the etiology of infection, inflammatory diseases including rheumatoid arthritis and atherosclerosis, as well as neuropathologies, particularly stroke, and Alzheimer's.

Evidence of platelet involvement in MS has recently emerged following demonstration of chronic platelet activation in peripheral blood of patients, as well as expression of the platelet-specific receptor CD41 in microarrays of chronic lesions. However, there is conflicting evidence whether the role of platelets in CNS inflammation is essential or contributory at all.

Accordingly, there is a need in the art for improved treatments for inflammatory neurological diseases, such as MS.

### SUMMARY

In producing the present invention, the inventors' studied the effects of inhibiting platelet accumulation in a murine experimental autoimmune encephalomyelitis (EAE) model of multiple sclerosis (MS). The inventors investigated whether platelets contribute to, or are essential to the development of neuroinflammation based on their understanding of the role of platelets in other biological pathways. The inventors showed for the first time that administration of anti-platelet factor CD39 to an accepted model of MS was therapeutically effective.

The inventors further demonstrated that administering CD39 as part of a binding protein (scFv-CD39) which targets activated glycoprotein GPIIb/IIIa had additional therapeutic efficacy, permitting administration at a lower dose and minimising side effects of administration. The inventors hypothesise that a binding protein comprising CD39 acts by enriching CD39 to activated platelets at the site of activated glycoprotein GPIIb/IIIa, and inhibits ligand binding to GPIIb/IIIa.

The findings by the inventors provide the basis for a binding protein comprising an extracellular domain of CD39 and a binding region that specifically binds to activated glycoprotein (GP)IIb/IIIa.

In one example, the extracellular domain of CD39 comprises or consists of a sequence set forth in SEQ ID NO: 4. For example, the extracellular domain of CD39 comprises a sequence set forth in SEQ ID NO: 4. In another example, the extracellular domain of CD39 consists of a sequence set forth in SEQ ID NO: 4.

In one example, the binding region specifically binds an epitope on GPIIb/IIIa recognised by a scFv consisting of a sequence set forth in SEQ ID NO: 1. For example, the binding region specifically binds the same epitope on GPIIb/IIIa that is bound by a scFv consisting of a sequence set forth in SEQ ID NO: 1.

In one example, the binding region competitively inhibits the binding of a scFv consisting of a sequence set forth in SEQ ID NO: 1 to an epitope on GPIIb/IIIa.

In one example, the binding region does not bind to inactive GPIIb/IIIa.

In one example, the binding region that specifically binds to activated GPIIb/IIIa inhibits GPIIb/IIIa receptor function and/or activity of GPIIb/IIIa. In one example, the binding region binds to activated GPIIb/IIIa and neutralizes GPIIb/IIIa receptor function and/or activity. In one example, the binding region that specifically binds to activated GPIIb/IIIa is a direct inhibitor of GPIIb/IIIa receptor function and/or activity. In one example, GPIIb/IIIa receptor function and/or activity is inhibited by at least about 20%. For example, the GPIIb/IIIa receptor function and/or activity is inhibited by about 30%, or about 40%, or about 50%, or about 60%, or about 70%, or about 80%, or about 85%, or about 90%, or about 95%, or about 99%, or about 100%. Methods for determining GPIIb/IIIa receptor function and/or activity are known in the art and/or described herein.

In one example, the binding region comprises an antibody variable region that binds to or specifically binds to GPIIb/IIIa and neutralizes GPIIb/IIIa receptor function and/or activity.

In one example, the binding region comprises an antibody variable region that binds to or specifically binds to activated GPIIb/IIIa. In one example, the binding region is a protein comprising a variable region fragment (Fv). For example, the protein is selected from the group consisting of
(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)2;
(viii) a Fv;
(ix) one of (i) to (viii) linked to a constant region of an antibody, Fc or a heavy chain constant domain (C_{H}) 2 and/or C_{H}3; or
(xi) an antibody.

In one example, the protein is an antibody or antigen binding fragment.

In one example, the binding region is a protein comprising a single chain Fv fragment (scFv).

In one example, the binding region is a protein that is recombinant, chimeric, CDR grafted, humanized, synhumanized, primatized, deimmunized or human. For example, the protein is human.

In one example, the binding protein is a fusion protein. For example, the binding protein is a fusion protein comprising an extracellular domain of CD39 and a binding region that specifically binds to activated GPIIb/IIIa.

In one example, the binding protein comprises a sequence which is at least 90% identical to a sequence set forth in SEQ ID NO: 1. For example, the binding protein comprises a sequence which is at least 95% identical to a sequence set forth in SEQ ID NO: 1. In another example, the binding protein comprises a sequence which is at least 98% identical to a sequence set forth in SEQ ID NO: 1. In a further example, the binding protein comprises a sequence which is at least 99% identical to a sequence set forth in SEQ ID NO: 1.

In one example, the binding protein comprises a sequence set forth in SEQ ID NO: 1. In one example, the binding protein is a fusion protein comprising a sequence set forth in SEQ ID NO: 1.

In one example, the binding protein comprises a binding region that comprises a sequence which is at least 90% identical to a sequence set forth in SEQ ID NO: 1. For example, the binding region comprises a sequence which is at least 95% identical to a sequence set forth in SEQ ID NO: 1. In another example, the binding region comprises a sequence which is at least 98% identical to a sequence set forth in SEQ ID NO: 1. In a further example, the binding region comprises a sequence which is at least 99% identical to a sequence set forth in SEQ ID NO: 1. In one example, the binding region comprises a sequence set forth in SEQ ID NO: 1. In one example, the binding region consists of a sequence set forth in SEQ ID NO: 1.

In one example, the binding protein comprises a sequence which is at least 90% identical to a sequence set forth in SEQ ID NO: 23. For example, the binding protein comprises a sequence which is at least 95% identical to a sequence set forth in SEQ ID NO: 23. In another example, the binding protein comprises a sequence which is at least 98% identical to a sequence set forth in SEQ ID NO: 23. In a further example, the binding protein comprises a sequence which is at least 99% identical to a sequence set forth in SEQ ID NO: 23.

In one example, the binding protein comprises a sequence set forth in SEQ ID NO: 23. In one example, the binding protein is a fusion protein comprising a sequence set forth in SEQ ID NO: 23.

In one example, the binding protein comprises a binding region that comprises a sequence which is at least 90% identical to a sequence set forth in SEQ ID NO: 23. For example, the binding region comprises a sequence which is at least 95% identical to a sequence set forth in SEQ ID NO: 23. In another example, the binding region comprises a sequence which is at least 98% identical to a sequence set forth in SEQ ID NO: 23. In a further example, the binding region comprises a sequence which is at least 99% identical to a sequence set forth in SEQ ID NO: 23. In one example, the binding region comprises a sequence set forth in SEQ ID NO: 23. In one example, the binding region consists of a sequence set forth in SEQ ID NO: 23.

In one example, the binding protein comprises a heavy chain variable region (V_{H}) comprising a sequence which is at least 90% identical to a sequence set forth in SEQ ID NO: 2 and a light chain variable region (V_{L}) comprising a sequence which is at least 90% identical to a sequence set forth in SEQ ID NO: 3.

In one example, the binding protein comprises a V_{H} comprising a sequence which is at least 90% identical to a sequence set forth in SEQ ID NO: 2, for example, a V_{H} comprising a sequence which is about 90%, or about 95%, or about 98% or about 99% identical to a sequence set forth in SEQ ID NO: 2. In one example, the binding protein comprises a V_{H} comprising a sequence set forth in SEQ ID NO: 2.

In one example, the binding protein comprises a V_{L} comprising a sequence which is at least 90% identical to a sequence set forth in SEQ ID NO: 3, for example, a V_{L} comprising a sequence which is about 90%, or about 95%, or about 98% or about 99% identical to a sequence set forth in SEQ ID NO: 3. In one example, the binding protein comprises a V_{L} comprising a sequence set forth in SEQ ID NO: 3.

In one example, the binding protein comprises a V_{H} comprising a sequence set forth in SEQ ID NO: 2 and a V_{L} comprising a sequence set forth in SEQ ID NO: 3.

In one example, the binding protein is a fusion protein comprising a V_{H} comprising a sequence set forth in SEQ ID NO: 2 and a V_{L} comprising a sequence set forth in SEQ ID NO: 3.

In one example, the binding region comprises a V_{H} comprising a sequence which is at least 90% identical to a sequence set forth in SEQ ID NO: 2 and a V_{L} comprising a sequence which is at least 90% identical to a sequence set forth in SEQ ID NO: 3.

In one example, the binding region comprises a V_{H} comprising a sequence which is at least 90% identical to a sequence set forth in SEQ ID NO: 2, for example, a V_{H} comprising a sequence which is about 90%, or about 95%, or about 98% or about 99% identical to a sequence set forth in SEQ ID NO: 2. In one example, the binding region comprises a V_{H} comprising a sequence set forth in SEQ ID NO: 2. In one example, the binding region comprises a V_{H} consisting of a sequence set forth in SEQ ID NO: 2.

In one example, the binding region comprises a V_{L} comprising a sequence which is at least 90% identical to a sequence set forth in SEQ ID NO: 3, for example, a V_{L} comprising a sequence which is about 90%, or about 95%, or about 98% or about 99% identical to a sequence set forth in SEQ ID NO: 3. In one example, the binding region comprises a V_{L} comprising a sequence set forth in SEQ ID NO: 3. In one example, the binding region comprises a V_{L} consisting of a sequence set forth in SEQ ID NO: 3.

In one example, the binding protein comprises a heavy chain variable region (V_{H}) comprising a sequence which is at least 90% identical to a sequence set forth in SEQ ID NO: 21 and a light chain variable region (V_{L}) comprising a sequence which is at least 90% identical to a sequence set forth in SEQ ID NO: 22.

In one example, the binding protein comprises a V_{H} comprising a sequence which is at least 90% identical to a sequence set forth in SEQ ID NO: 21, for example, a V_{H} comprising a sequence which is about 90%, or about 95%, or about 98% or about 99% identical to a sequence set forth in SEQ ID NO: 21. In one example, the binding protein comprises a V_{H} comprising a sequence set forth in SEQ ID NO: 21.

In one example, the binding protein comprises a V_{L} comprising a sequence which is at least 90% identical to a sequence set forth in SEQ ID NO: 22, for example, a V_{L} comprising a sequence which is about 90%, or about 95%, or about 98% or about 99% identical to a sequence set forth in SEQ ID NO: 22. In one example, the binding protein comprises a V_{L} comprising a sequence set forth in SEQ ID NO: 22.

In one example, the binding protein comprises a V_{H} comprising a sequence set forth in SEQ ID NO: 21 and a V_{L} comprising a sequence set forth in SEQ ID NO: 22.

In one example, the binding protein is a fusion protein comprising a V_{H} comprising a sequence set forth in SEQ ID NO: 21 and a V_{L} comprising a sequence set forth in SEQ ID NO: 22.

In one example, the binding region comprises a V_{H} comprising a sequence which is at least 90% identical to a sequence set forth in SEQ ID NO: 21 and a V_{L} comprising a sequence which is at least 90% identical to a sequence set forth in SEQ ID NO: 22.

In one example, the binding region comprises a V_{H} comprising a sequence which is at least 90% identical to a sequence set forth in SEQ ID NO: 21, for example, a V_{H} comprising a sequence which is about 90%, or about 95%, or about 98% or about 99% identical to a sequence set forth in SEQ ID NO: 21. In one example, the binding region comprises a V_{H} comprising a sequence set forth in SEQ ID NO: 21. In one example, the binding region comprises a V_{H} consisting of a sequence set forth in SEQ ID NO: 21.

In one example, the binding region comprises a V_{L} comprising a sequence which is at least 90% identical to a sequence set forth in SEQ ID NO: 22, for example, a V_{L} comprising a sequence which is about 90%, or about 95%, or about 98% or about 99% identical to a sequence set forth in SEQ ID NO: 22. In one example, the binding region comprises a V_{L} comprising a sequence set forth in SEQ ID NO: 22. In one example, the binding region comprises a V_{L} consisting of a sequence set forth in SEQ ID NO: 22.

In one example, the binding protein comprises a variable region comprising the complementary determining regions (CDRs) of the V_{H} and/or the V_{L} of SEQ ID NO: 2 and SEQ ID NO: 3. For example, the binding protein comprises the CDRs of the V_{H} of SEQ ID NO: 2 and the CDRs of the V_{L} of SEQ ID NO: 3.

In one example, the binding protein comprises:
(a) a V_{H} comprising:
   (i) a CDR1 comprising a sequence set forth in SEQ ID NO: 15;
   (ii) a CDR2 comprising a sequence set forth in SEQ ID NO: 16; and
   (iii) a CDR3 comprising a sequence set forth in SEQ ID NO: 17; and
(b) a V_{L} comprising:
   (i) a CDR1 comprising a sequence set forth in SEQ ID NO: 18;
   (ii) a CDR2 comprising a sequence set forth in SEQ ID NO: 19; and
   (iii) a CDR3 comprising a sequence set forth in SEQ ID NO: 20.

In one example, the extracellular domain of CD39 is linked to the binding region. In one example, the extracellular domain of CD39 is linked to the binding region directly (i.e., without a linking region). In another example, the extracellular domain of CD39 is linked to the binding region via a linker. In one example, the linker is a peptide linker comprising between 3 and 30 amino acids in length. In one example, the linker comprises a sequence AlaAlaAla.

In one example, the binding protein of the present disclosure comprises a sequence which is at least 70% identical to the sequence set forth in SEQ ID NO: 6. In one example, the binding protein comprises a sequence which is at least 80% identical to the sequence set forth in SEQ ID NO: 6. In one example, the binding protein comprises a sequence which is at least 90% identical to the sequence set forth in SEQ ID NO: 6. In one example, the binding protein comprises a sequence which is at least 95% identical to the sequence set forth in SEQ ID NO: 6.

In one example, the binding protein of the present disclosure comprises a sequence set forth in SEQ ID NO: 6.

In one example, the binding protein consists of a sequence set forth in SEQ ID NO: 6.

In one example, the binding protein of the present disclosure comprises a sequence which is at least 70% identical to the sequence set forth in SEQ ID NO: 24. In one example, the binding protein comprises a sequence which is at least 80% identical to the sequence set forth in SEQ ID NO: 24. In one example, the binding protein comprises a sequence which is at least 90% identical to the sequence set forth in SEQ ID NO: 24. In one example, the binding protein comprises a sequence which is at least 95% identical to the sequence set forth in SEQ ID NO: 24.

In one example, the binding protein of the present disclosure comprises a sequence set forth in SEQ ID NO: 24.

In one example, the binding protein consists of a sequence set forth in SEQ ID NO: 24.

In one example, the binding protein of the present disclosure comprises a sequence which is at least 70% identical to the sequence set forth in SEQ ID NO: 25. In one example, the binding protein comprises a sequence which is at least 80% identical to the sequence set forth in SEQ ID NO: 25. In one example, the binding protein comprises a sequence which is at least 90% identical to the sequence set forth in SEQ ID NO: 25. In one example, the binding protein comprises a sequence which is at least 95% identical to the sequence set forth in SEQ ID NO: 25.

In one example, the binding protein of the present disclosure comprises a sequence set forth in SEQ ID NO: 25.

In one example, the binding protein consists of a sequence set forth in SEQ ID NO: 25.

In one example, the binding protein of the present disclosure comprises a sequence which is at least 70% identical to the sequence set forth in SEQ ID NO: 26. In one example, the binding protein comprises a sequence which is at least 80% identical to the sequence set forth in SEQ ID NO: 26. In one example, the binding protein comprises a sequence which is at least 90% identical to the sequence set forth in SEQ ID NO: 26. In one example, the binding protein comprises a sequence which is at least 95% identical to the sequence set forth in SEQ ID NO: 26.

In one example, the binding protein of the present disclosure comprises a sequence set forth in SEQ ID NO: 26.

In one example, the binding protein consists of a sequence set forth in SEQ ID NO: 26.

In one example, the binding protein of the present disclosure comprises a sequence which is at least 70% identical to the sequence set forth in SEQ ID NO: 27. In one example, the binding protein comprises a sequence which is at least 80% identical to the sequence set forth in SEQ ID NO: 27. In one example, the binding protein comprises a sequence which is at least 90% identical to the sequence set forth in SEQ ID NO: 27. In one example, the binding protein comprises a sequence which is at least 95% identical to the sequence set forth in SEQ ID NO: 27.

In one example, the binding protein of the present disclosure comprises a sequence set forth in SEQ ID NO: 27.

In one example, the binding protein consists of a sequence set forth in SEQ ID NO: 27

In one example, the binding protein of the present disclosure comprises a sequence which is at least 70% identical to the sequence set forth in SEQ ID NO: 28. In one example, the binding protein comprises a sequence which is at least 80% identical to the sequence set forth in SEQ ID NO: 28. In one example, the binding protein comprises a sequence which is at least 90% identical to the sequence set forth in SEQ ID NO: 28. In one example, the binding protein comprises a sequence which is at least 95% identical to the sequence set forth in SEQ ID NO: 28.

In one example, the binding protein of the present disclosure comprises a sequence set forth in SEQ ID NO: 28.

In one example, the binding protein consists of a sequence set forth in SEQ ID NO: 28.

In some examples, the binding protein further comprises an albumin sequence. In some examples, the albumin sequence is positioned C-terminal to the binding region in the binding protein. In some examples, the albumin sequence is positioned N-terminal to the binding region in the binding protein. In some examples, the albumin sequence is positioned C-terminal to the extracellular domain of CD39 in the binding protein. In some examples, the albumin sequence is positioned N-terminal to the extracellular domain of CD39 in the binding protein.

In some examples, the albumin sequence is positioned C-terminal to the binding region and N-terminal to the extracellular domain of CD39 in the binding protein. Thus, in some examples, the albumin sequence is positioned between the binding region and the extracellular domain of CD39.

In some examples, the albumin is human serum albumin.

In an example, the human serum albumin comprises a sequence which is at least 70% identical to a sequence set forth in SEQ ID NO: 31. In an example, the human serum albumin comprises a sequence which is at least 80% identical to a sequence set forth in SEQ ID NO: 31. In an example, the human serum albumin comprises a sequence which is at least 90% identical to a sequence set forth in SEQ ID NO: 31. In an example, the human serum albumin comprises a sequence which is at least 95% identical to a sequence set forth in SEQ ID NO: 31. In an example, the human serum albumin comprises a sequence which is at least 99% identical to a sequence set forth in SEQ ID NO: 31.

In an example, the human serum albumin comprises a sequence set forth in SEQ ID NO: 31. In an example, the human serum albumin consists of a sequence set forth in SEQ ID NO: 31.

In one example, the binding protein of the present disclosure comprises a sequence which is at least 70% identical to the sequence set forth in SEQ ID NO: 32. In one example, the binding protein comprises a sequence which is at least 80% identical to the sequence set forth in SEQ ID NO: 32. In one example, the binding protein comprises a sequence which is at least 90% identical to the sequence set forth in SEQ ID NO: 32. In one example, the binding protein comprises a sequence which is at least 95% identical to the sequence set forth in SEQ ID NO: 32.

In one example, the binding protein of the present disclosure comprises a sequence set forth in SEQ ID NO: 32. In one example, the binding protein of the present disclosure consists of a sequence set forth in SEQ ID NO: 32.

In one example, the binding protein of the present disclosure comprises a sequence which is at least 70% identical to the sequence set forth in SEQ ID NO: 33. In one example, the binding protein comprises a sequence which is at least 80% identical to the sequence set forth in SEQ ID NO: 33. In one example, the binding protein comprises a sequence which is at least 90% identical to the sequence set forth in SEQ ID NO: 33. In one example, the binding protein comprises a sequence which is at least 95% identical to the sequence set forth in SEQ ID NO: 33.

In one example, the binding protein of the present disclosure comprises a sequence set forth in SEQ ID NO: 33. In one example, the binding protein of the present disclosure consists of a sequence set forth in SEQ ID NO: 33.

In one example, the binding protein of the present disclosure comprises a sequence which is at least 70% identical to the sequence set forth in SEQ ID NO: 34. In one example, the binding protein comprises a sequence which is at least 80% identical to the sequence set forth in SEQ ID NO: 34. In one example, the binding protein comprises a sequence which is at least 90% identical to the sequence set forth in SEQ ID NO: 34. In one example, the binding protein comprises a sequence which is at least 95% identical to the sequence set forth in SEQ ID NO: 34.

In one example, the binding protein of the present disclosure comprises a sequence set forth in SEQ ID NO: 34. In one example, the binding protein of the present disclosure consists of a sequence set forth in SEQ ID NO: 34.

In one example, the binding protein of the present disclosure comprises a sequence which is at least 70% identical to the sequence set forth in SEQ ID NO: 35. In one example, the binding protein comprises a sequence which is at least 80% identical to the sequence set forth in SEQ ID NO: 35. In one example, the binding protein comprises a sequence which is at least 90% identical to the sequence set forth in SEQ ID NO: 35. In one example, the binding protein comprises a sequence which is at least 95% identical to the sequence set forth in SEQ ID NO: 35.

In one example, the binding protein of the present disclosure comprises a sequence set forth in SEQ ID NO: 35. In one example, the binding protein of the present disclosure consists of a sequence set forth in SEQ ID NO: 35.

The present disclosure also provides a composition comprising a binding protein of the disclosure and a pharmaceutically acceptable carrier. In one example, the binding protein of the present disclosure is within a composition. For example, the composition comprises a fusion protein comprising an extracellular domain of CD39 and a binding region that specifically binds to activated GPIIb/IIIa as described herein.

The findings by the inventors also provide the basis for a method of treating or preventing an inflammatory neurological disease in a subject, the method comprising administering to the subject a protein comprising or consisting of an extracellular domain of CD39.

In one example, the protein comprising the extracellular domain of CD39 is administered in the form of a binding protein. For example, the protein is a binding protein. In one example, example, the disclosure provides a method of treating or preventing an inflammatory neurological disease in a subject, the method comprising administering to the subject a binding protein, wherein the binding protein comprises an extracellular domain of CD39.

In one example, the present disclosure provides a method of treating an inflammatory neurological disease in a subject, the method comprising administering to the subject a binding protein, wherein the binding protein comprises (a) an extracellular domain of CD39; and (b) a binding region that specifically binds to activated glycoprotein (GP)IIb/IIIa.

In another example, the present disclosure provides a method of preventing an inflammatory neurological disease in a subject, the method comprising administering to the subject a binding protein, wherein the binding protein comprises (a) an extracellular domain of CD39; and (b) a binding region that specifically binds to activated glycoprotein (GP)IIb/IIIa.

In one example, the inflammatory neurological disease is selected from the group consisting of multiple sclerosis, systemic lupus erythematosus (SLE), Guillain-Barre syndrome, Lambert-Eaton myasthenic syndrome, myasthenia gravis, transverse myelitis, leukodystrophy and progressive multifocal leukoencephalopathy.

In one example, the inflammatory neurological disease is multiple sclerosis.

In one example, the inflammatory neurological disease is systemic lupus erythematosus (SLE).

In one example, the inflammatory neurological disease is Guillain-Barre syndrome.

In one example, the inflammatory neurological disease is Lambert-Eaton myasthenic syndrome.

In one example, the inflammatory neurological disease is myasthenia gravis.

In one example, the inflammatory neurological disease is transverse myelitis.

In one example, the inflammatory neurological disease is leukodystrophy.

In one example, the inflammatory neurological disease is progressive multifocal leukoencephalopathy.

In one example, the inflammatory neurological disease is a degenerative disease of the central nervous system. For example, the degenerative disease of the central nervous system is multiple sclerosis (MS).

In one example, the binding protein is administered in an amount to increase the level of circulating anti-platelet factor CD39. For example, the circulating or systemic level of anti-platelet factor CD39 is increased by at least about 10%. For example, the level of anti-platelet factor CD39 is increased by about 10%, or about 20%, or about 25%, or about 30%, or about 40%, or about 50%, or about 60%, or about 70%, or about 80%, or about 90%, or about 100%. In one example, the circulating or systemic level of anti-platelet factor CD39 is increased by at least about 10% compared to the level before administration of the anti-platelet factor CD39. In one example, the circulating or systemic level of anti-platelet factor CD39 is increased by at least about 10% compared to a control level.

The present disclosure also provides a method of treating or preventing an inflammatory neurological disease in a subject comprising administration of the binding protein of the present disclosure or the composition comprising a binding protein of the present disclosure to the subject.

In one example, the disclosure provides use of binding protein of the present disclosure or the composition comprising a binding protein of the present disclosure in the manufacture of a medicament for the treatment or prevention of an inflammatory neurological disease in a subject. In one example, the subject is in need of treatment (i.e., in need thereof).

In one example, the inflammatory neurological disease is multiple sclerosis (MS). In one example, the subject suffers from an inflammatory neurological disease. For example, the subject suffers from multiple sclerosis. In one example, the subject has been diagnosed as suffering from MS. In one example, the subject is receiving treatment for MS.

In one example of any method described herein, the binding protein of the present disclosure is administered before or after the development of MS. In one example, the binding protein of the present disclosure is administered before the development of the MS. In one example, the binding protein of the present disclosure is administered after the development of the MS.

In one example, the subject is at risk of developing MS or a symptom thereof. An exemplary subject at risk of developing MS suffers from thyroid disease, type I diabetes and/or inflammatory bowel disease.

Additional or alternative characteristics of a subject at risk of developing MS include one or more of the following characteristics:
is aged between 15 and 60;
is female;
has a family history of MS;
suffers from a viral infection (e.g., Epstein-Barr); and/or
is a smoker.

In one example, the binding protein is administered before or after the onset of MS and/or a symptom of MS. For example, the binding protein is administered prophylactically or therapeutically. In one example, the binding protein is administered prior to the onset of (clinical) symptoms of MS. For example, the binding protein is administered to the subject prophylactically. In one example, the binding protein is administered after the onset of symptoms of MS. For example, the binding protein is administered to the subject therapeutically. In one example, the binding protein of the present disclosure is administered at a dose that alleviates or reduces one or more of the symptoms of MS.

Symptoms of MS will be apparent to the skilled person and include, for example:
Motor control, including, for example, muscular spasms and problems with weakness, coordination, balance and functioning of the arms and legs;
Fatigue, including heat sensitivity;
Other neurological symptoms, including, for example, vertigo, pins and needles, neuralgia and visual disturbances;
Continence problems, including bladder incontinence and constipation; and/or
Neuropsychological symptoms, including, for example, memory loss, depression and cognitive difficulties.

In one example, the onset of symptoms is characterised by:
an increase in circulating platelet numbers;
an increase in plasma levels of adenosine-5′-diphosphate (ADP);
an increase in platelet accumulation and/or platelet infiltration in the CNS parenchyma;
an increase in astrocytic and/or microglial glial reactivity;
an increase in demyelination; and/or
an increase in lymphocytic infiltration.

In one example, the onset of symptoms is characterised by an increase in circulating platelet numbers in the subject.

Methods for assessing each of the foregoing are known in the art and/or described herein.

In one example, the binding protein is administered in an amount effective to:
a) decrease plasma levels of adenosine-5′-diphosphate (ADP); adenosine-5′-triphosphate (ATP)
b) reduce and/or prevent platelet accumulation and/or platelet infiltration in the CNS parenchyma;
c) reduce and/or prevent astrocytic and/or microglial glial reactivity;
d) reduce and/or prevent demyelination; and/or
e) reduce and/or prevent lymphocytic infiltration
f) provide strong anti-inflammatory effects.

In one example of any method described herein, the subject is a mammal, for example a primate such as a human.

Methods of treatment described herein can additionally comprise administering a further compound to reduce, treat or prevent the effect of the MS.

The present disclosure also provides a composition comprising a protein of the present disclosure for use in treating or preventing an inflammatory neurological disease (e.g., MS).

The present disclosure also provides a composition comprising a binding protein of the present disclosure for use in treating or preventing an inflammatory neurological disease (e.g., MS).

The present disclosure also provides use of a composition comprising a protein of the present disclosure in the manufacture of a medicament for treating or preventing an inflammatory neurological disease (e.g., MS).

The present disclosure also provides use of a composition comprising a binding protein of the present disclosure in the manufacture of a medicament for treating or preventing an inflammatory neurological disease (e.g., MS).

In one example, the disclosure provides use of a protein in the manufacture of a medicament for treating or preventing an inflammatory neurological disease in a subject, wherein the protein comprises an extracellular domain of CD39.

In one example, the disclosure provides use of a binding protein in the manufacture of a medicament for treating or preventing an inflammatory neurological disease in a subject, wherein the binding protein comprises a) an extracellular domain of CD39; and b) a binding region that specifically binds to activated glycoprotein (GP)IIb/IIIa.

The present disclosure also provides a kit comprising at least one protein that of the present disclosure packaged with instructions for use in treating or preventing an inflammatory neurological disease (e.g., MS) in a subject. Optionally, the kit additionally comprises a therapeutically active compound or drug.

The present disclosure also provides a kit comprising at least one binding protein that of the present disclosure packaged with instructions for use in treating or preventing an inflammatory neurological disease (e.g., MS) in a subject. Optionally, the kit additionally comprises a therapeutically active compound or drug.

The present disclosure also provides a kit comprising at least one protein of the present disclosure packaged with instructions to administer the binding protein to a subject who is suffering from or at risk of suffering from an inflammatory neurological disease (e.g., MS), optionally, in combination with a therapeutically active compound or drug.

The present disclosure also provides a kit comprising at least one binding protein of the present disclosure packaged with instructions to administer the binding protein to a subject who is suffering from or at risk of suffering from an inflammatory neurological disease (e.g., MS), optionally, in combination with a therapeutically active compound or drug.

Exemplary effects of binding proteins of the present disclosure are described herein and are to be taken to apply mutatis mutandis to the examples of the disclosure set out in the previous nine paragraphs.

### BRIEF DESCRIPTION OF THE DRAWINGS

FIG. 1 is a graphical representation showing circulating platelet numbers over the progression of EAE in platelet depleted and control groups. **, p≤0.01; Error bars represent SEM where n≤3.

FIG. 2 is a graphical representation showing the effect of platelet depletion on clinical profile and lesion development. (a-b): R300 or C301 preparations were administered to MOG₃₅₋₅₅-induced and vehicle-only groups, over the 2-6, 7-11 and 11-15 dpi (3 injections) periods (coloured arrows and boxes) and clinical scores (a) and percentage weight loss (b) recorded. (**=p<0.01, ***=p<0.001). Error bars=mean±SEM, n=8/group. (c-d) Parenchymal accumulation of CD41 and CD3 in MOG₃₅₋₅₅-induced/no further treatment group, from 8-16 dpi using vehicle-only and MOG₃₅₋₅₅-induced/R300 treated mice as control. Expression fold-change values relative to vehicle-only.

FIG. 3 is a graphical representation showing the effect of platelet function inhibition on clinical profile. (a) Effect of scFv-CD39 or scFvₘᵤₜ-CD39 antibody administration over 7-15 dpi (5 injections) and clinical scores and percentage weight loss recorded. Error bars=mean±SEM, n=4/group. Statistical significance indicated by, *=p<0.05. (b) Table showing a summary of the mean values of parameters being compared between groups. A denotes values from one mouse only (not mean values).

FIG. 4 is a graphical representation showing relationship between platelet accumulation, MOG₃₅₋₅₅-CD4⁺ cell accumulation and EAE development. *=p<0.05; **=p<0.01; n=6/group/time point. (A) Platelet counts over disease course and antigen-specific T cell accumulation expressed as % MOG₃₅₋₅₅-CD4⁺ over total CD4⁺ cells. (B) shows inhibition of MOG₃₅₋₅₅-CD4⁺ cell accumulation by platelet depletion. (C) shows effect of platelet depletion on disease course and weight loss. (D) shows confirmation of platelet depletion with anti-CD42b and estimation of sP-selection by ELISA. (Experiments B-D n=6 mice/group/time point). *=p<0.05; **=p<0.01.

FIG. 5 is a graphical representation showing the effect of platelet depletion on inflammatory infiltration into the (A) hippocampus and (B) spinal cord. n=4 mice/group×4 sections/mouse. **=p<0.01.

FIG. 6 is a graphical representation showing the effect of platelet depletion on anxiety-like behavior and the pro-inflammatory environment in the hippocampus. (A) EPM evaluation (n=8/group). (B) (n=4/group) shows the qPCR analysis of pro-inflammatory cytokines TNF-α, IFN-γ and platelet marker CD41. (C) shows the disease profile in mice used in the EPM test. *=p<0.05; **=p<0.01.

FIG. 7 is a graphical representation showing quantification of platelet entry into the hippocampus and hallmarks of neuroinflammation. (A) CD42b (platelets), (B) Ibal (microglial reactivity) and (C) CD3 (T lymphocytes). n=4 mice/group×6 sections/mouse. **=p<0.01.

FIG. 8 is a graphical representation showing the effect of scFv-CD39 and scFvₘᵤₜ-CD39 treatment on disease severity and progression in all clinical measures, including clinical score over time (A), cumulative scores over time (B), survival curve (C) and weight loss over time (D).

FIG. 9 is a graphical representation showing the effect on clinical scores of scFv-CD39 treatment (targ CD39, B) compared to scFvₘᵤₜ-CD39 treatment (non-targ CD39, A) 7 days post disease induction and scFv-CD39 treatment 12 days post disease induction (i.e., after disease onset).

FIG. 10 is a graphical representation showing the effect on weight loss (A) between EAE-induced and untreated mice, scFv-CD39 treatment, scFvₘᵤₜ-CD39 treatment, and normal mice. The effects on clinical scores are shown for prophylactic treatment from 7 dpi onwards (B) and therapeutic treatment from 12 dpi onwards (C)

FIG. 11 is graphical representation of the clinical scores observed for the standard EAE disease model (SP) and the mild EAE disease model (MP). Data plotted as ±SEM, in each group n=14 (MP) n=8 (SP). Significance was generated using a one-tailed Student's t-test assuming unequal variance. When the mild protocol (MP) was compared to the standard protocol (SP), p-value obtained was significant (p=0.003).

FIG. 12 is a series of micrographs demonstrating evidence of pathological hallmarks of EAE in the mild EAE model described in Example 10. (A) to (C) show the dissection protocol, with the red box in (C), showing the region of the spinal cord where images (D)-(K) are taken from. (D)-(F) show the presence of immune cells highlighted with the white arrows, which are not detectable in the control tissues (G). (H)-(J) show areas characterized by loss of myelin highlighted with the arrows, in contrast to the evenly distributed myelin presence in the control tissue (K).

FIG. 13 is a graphical comparison of clinical score profiles using the mild EAE disease model. The clinical scores of scFv-CD39 treated mice (TargCD39), scFvₘᵤₜ-CD39 treated mice (Non-targCD39) and untreated mice (EAE) are shown. Drug treatment was initiated when mice reached a clinical score>1, <2 and a minimum of 10-12% weight loss (arrow).

FIG. 14 is a graphical representation of Kaplan-Meier survival curves for scFv-CD39 treated mice (Targ; sample size of 9), scFvₘᵤₜ-CD39 treated mice (Non-Targ; sample size of 8) and untreated mice (EAE; sample size of 10). The mild EAE disease model was used and drug treatment was initiated when mice reached a clinical score>1, <2 and a minimum of 10-12% weight loss.

FIG. 15 is a graphical representation of individual mouse clinical score profiles of scFv-CD39 treated mice. The mild EAE disease model was used and drug treatment was initiated when mice reached a clinical score>1, <2 and a minimum of 10-12% weight loss.

FIG. 16 is a graphical representation of flow cytometric assays of scFv-CD39 (targ-CD39) and scFvₘᵤₜ-CD39 (non-targ-CD39) binding to human platelets, detected with an Alexa Fluor 488-coupled anti-Penta-His antibody that binds to the constructs' 6xHis-tag. Bar graphs depict the median fluorescence intensity values of 3 independent experiments (***P, 0.001). These assays were analyzed with 2-way repeated-measures analysis of variance with a Bonferroni post-test.

FIG. 17 a graphical representation of flow cytometric assays to determine the ability of scFv-HSA-CD39 to bind to activated platelets. scFv-HSA-CD39 bound to activated platelets (B) but not to non-activated platelets (A).

FIG. 18 a graphical representation of flow cytometric assays to determine the ability of scFv-HSA-CD39 to hydrolyse ADP and prevent platelet activation. PAC-1 FITC (a fluorescently labelled antibody that also binds to GPIIb/IIIa on activated platelets) did not bind to non-activated human platelets (A) but was able to bind to platelets activated with 20 nM ADP (B) in the absence of scFv-HSA-CD39. In (C), scFv-HSA-CD39 successfully hydrolysed 20 nM ADP thereby preventing activation of the platelets and PAC-1 FITC binding.

### KEY TO SEQUENCE LISTING

SEQ ID NO: 1 amino acid sequence of single-chain (scFv) antibody against platelet marker CD41
SEQ ID NO: 2 heavy chain V_{H} amino acid sequence of scFv to CD41
SEQ ID NO: 3 light chain V_{L} amino acid sequence of scFv to CD41
SEQ ID NO: 4 amino acid sequence of extracellular domain of CD39
SEQ ID NO: 5 nucleotide sequence of scFv-CD39
SEQ ID NO: 6 amino acid sequence of scFv-CD39
SEQ ID NO: 7 amino acid sequence of scFVmut
SEQ ID NO: 8 amino acid sequence of human CD39
SEQ ID NO: 9 heavy chain V_{H} amino acid sequence of scFvₘᵤₜ to CD41
SEQ ID NO: 10 light chain V_{L} amino acid sequence of scFvₘᵤₜ to CD41
SEQ ID NO: 11 amino acid sequence of scFVmut-CD39
SEQ ID NO: 12 amino acid sequence of human GPIIb
SEQ ID NO: 13 amino acid sequence of human GPIIIa
SEQ ID NO: 14 nucleotide sequence of scFv-CD39mut
SEQ ID NO: 15 amino acid sequence of scFv V_{H} CDR1
SEQ ID NO: 16 amino acid sequence of scFv V_{H} CDR2
SEQ ID NO: 17 amino acid sequence of scFv V_{H} CDR3
SEQ ID NO: 18 amino acid sequence of scFv V_{L} CDR1
SEQ ID NO: 19 amino acid sequence of scFv V_{L} CDR2
SEQ ID NO: 20 amino acid sequence of scFv V_{L} CDR3
SEQ ID NO: 21 an alternative V_{H} amino acid sequence of a scFv to CD41
SEQ ID NO: 22 an alternative V_{L} amino acid sequence of a scFv to CD41
SEQ ID NO: 23 an alternative amino acid sequence of a scFv to CD41
SEQ ID NO: 24 an alternative amino acid sequence of scFv-CD39
SEQ ID NO: 25 an alternative amino acid sequence of scFv-CD39
SEQ ID NO: 26 an alternative amino acid sequence of scFv-CD39
SEQ ID NO: 27 an alternative amino acid sequence of scFv-CD39
SEQ ID NO: 28 an alternative amino acid sequence of scFv-CD39
SEQ ID NO: 29 an amino acid sequence of a flexible linker
SEQ ID NO: 30 an amino acid sequence of a leader sequence
SEQ ID NO: 31 an amino acid sequence of human serum albumin
SEQ ID NO: 32 an amino acid sequence of a scFv-CD39-HSA-CD39 fusion protein
SEQ ID NO: 33 an amino acid sequence of a scFv-CD39-HSA-CD39 fusion protein
SEQ ID NO: 34 an amino acid sequence of a scFv-CD39-HSA-CD39 fusion protein
SEQ ID NO: 35 an amino acid sequence of a scFv-CD39-HSA-CD39 fusion protein

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, antibodies and fragments thereof herein may be further clarified by the discussion in Kabat Sequences of Proteins of Immunological Interest, National Institutes of Health, Bethesda, Md., 1987 and 1991.

The term “EU numbering system of Kabat” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Reference herein to a range of, e.g., residues, will be understood to be inclusive. For example, reference to “a region comprising amino acids 56 to 65 of SEQ ID NO: 1” will be understood to mean that the region comprises a sequence of amino acids as numbered 56, 57, 58, 59, 60, 61, 62, 63, 64 and 65 in SEQ ID NO: 1.

Selected Definitions

CD39 (Cluster of Differentiation 39) also known as ectonucleoside triphosphate diphosphohydrolase-1 (gene: ENTPD1; protein: NTPDase1), is a cell surface-located enzyme with an extracellularly facing catalytic site. For the purposes of nomenclature only and not limitation an exemplary sequence of human CD39 is set out in NCBI Reference Sequence NM_001776.5 and SEQ ID NO: 8. Additional sequences of CD39 from other species can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)).

Glycoprotein IIb/IIIa (GPIIb/IIIa, also known as integrin (αIIbβ3) is an integrin complex found on platelets. It is a receptor for fibrinogen and von Willebrand factor and aids platelet activation. The GPIIb/IIIa complex is formed via calcium-dependent association of gpIIb and gpIIIa, a required step in normal platelet aggregation and endothelial adherence. Platelet activation by ADP (blocked by clopidogrel) leads to the aforementioned conformational change in platelet gpIIb/IIIa receptors that induces binding to fibrinogen. For the purposes of nomenclature only and not limitation an exemplary sequence of human GPIIb is set out in NCBI Gene ID: 3674, NCBI Reference Sequence: NG_008331.1 and SEQ ID NO: 12. For the purposes of nomenclature only and not limitation an exemplary sequence of human GPIIIa is set out in NCBI Gene ID: 3690, NCBI Reference Sequence: NG_008332.2 and SEQ ID NO: 13. Additional sequences of GPIIb and/or IIIa from other species can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)).

As used herein, the term “inflammatory neurological disease” shall be taken to include any disorder characterized by a defect in neuronal signaling and/or neuronal dysfunction and/or neuronal cell death resulting from an inflammatory response, and, in some examples, an autoimmune response. In one example, an inflammatory neurological disorder is a disorder associated with or caused by myelin degeneration and/or autoantibodies against a component of the nervous system, such as, for example a component of myelin or a phospholipid or a ganglioside.

The term “binding region” shall be understood to refer to a binding protein or part thereof or other region of the binding protein that is capable of interacting with or specifically binding to an antigen (e.g., a cell component or molecule, such as a protein, e.g., a glycoprotein). For example, the binding region can be an antibody or an antigen binding fragment of an antibody (e.g., a Fv or a scFv, etc.)

As used herein, the term “binds” in reference to the interaction of a binding region of a binding protein with GPIIb/IIIa means that the interaction is dependent upon the presence of a particular structure (e.g., epitope) on the component. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” shall be taken to mean that the binding interaction between the binding region on the binding protein and GPIIb/IIIa is dependent on the presence of the antigenic determinant or epitope. The binding region preferentially binds or recognizes a specific antigenic determinant or epitope even when present in a mixture of other molecules or organisms. In one example, the binding region reacts or associates more frequently, more rapidly, with greater duration and/or with greater affinity with the specific component or cell expressing same than it does with alternative antigens or cells. It is also understood by reading this definition that, for example, a binding region the specifically binds to a particular component may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. For example, a binding protein comprising the binding region of the disclosure is contacted with the component or a cell expressing same or a mutant form thereof or an alternative antigen. The binding to the component or mutant form or alternative antigen is then determined and a binding region that binds as set out above is considered to specifically bind to the component.

As used herein, the term “neutralize” shall be taken to mean that a protein is capable of blocking, reducing or preventing ligand binding (e.g. fibrinogen/fibrin) to GPIIb/IIIa. Methods for determining neutralization are known in the art and/or described herein.

As used herein, the term “epitope” (syn. “antigenic determinant”) shall be understood to mean a region of GPIIb/IIIa to which a protein comprising an antigen binding domain of an antibody binds. This term is not necessarily limited to the specific residues or structure to which the protein makes contact. For example, this term includes the region spanning amino acids contacted by the protein and/or at least 5 to 10 or 2 to 5 or 1 to 3 amino acids outside of this region. In some examples, the epitope is a linear series amino acids. An epitope may also comprise a series of discontinuous amino acids that are positioned close to one another when GPIIb/IIIa is folded, that is, a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics. An epitope or peptide or polypeptide comprising same can be administered to an animal to generate antibodies against the epitope.

The term “competitively inhibits” shall be understood to mean that a binding protein of the disclosure reduces or prevents binding of a recited antibody to GPIIb/IIIa, for example, to a scFv consisting of a sequence set forth in SEQ ID NO: 1. This may be due to the protein (or antigen binding domain) binding to the same or an overlapping epitope as the antibody. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Methods for determining competitive inhibition of binding are known in the art and/or described herein. For example, the antibody is exposed to GPIIb/IIIa either in the presence or absence of the protein. If less antibody binds in the presence of the protein than in the absence of the protein, the protein is considered to competitively inhibit binding of the antibody. In one example, the competitive inhibition of binding is caused by the antigen binding domain of the protein on GPIIb/IIIa overlapping with the antigen binding domain of the antibody.

“Overlapping” in the context of two epitopes means that two epitopes share a sufficient number of amino acid residues to permit a binding protein of the disclosure that binds to one epitope to competitively inhibit the binding of a recited antibody to GPIIb/IIIa that binds to the other epitope. For example, the “overlapping” epitopes share at least 1 or 2 or 3 or 4 or 5 or 6 or 7 or 8 or 9 or 10 or 11 or 12 or 13 or 14 or 15 or 16 or 17 or 18 or 19 or 20 amino acids.

The term “recombinant” shall be understood to mean the product of artificial genetic recombination. Accordingly, in the context of an antibody or antigen binding fragment thereof, this term does not encompass an antibody naturally occurring within a subject's body that is the product of natural recombination that occurs during B cell maturation. However, if such an antibody is isolated, it is to be considered an isolated protein comprising an antibody variable region. Similarly, if nucleic acid encoding the protein is isolated and expressed using recombinant means, the resulting protein is a recombinant protein. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a light chain variable region (V_{L}) and a polypeptide comprising a heavy chain variable region (V_{H}). An antibody also generally comprises constant domains, some of which can be arranged into a constant region, which includes a constant fragment or fragment crystallizable (Fc), in the case of a heavy chain. A V_{H} and a V_{L} interact to form an Fv comprising an antigen binding region that is capable of specifically binding to one or a few closely related antigens. Generally, a light chain from mammals is either a κ light chain or a λ light chain and a heavy chain from mammals is α, δ, ε, γ, or β. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies, synhumanized antibodies and chimeric antibodies.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”. According to the numbering system of Kabat, V_{H} FRs and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4).

“Framework regions” (hereinafter FR) are those variable domain residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an antibody, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

An “antigen binding fragment” of an antibody comprises one or more variable regions of an intact antibody. Examples of antibody fragments include Fab, Fab′, F(ab′)2 and Fv fragments; diabodies; linear antibodies; single-chain antibody molecules, half antibodies and multispecific antibodies formed from antibody fragments.

As used herein, the term “mutant” or “mutated” refers to a scFv (e.g., scFvₘᵤₜ) which has undergone modification (e.g., deletion or truncation) of one or more amino acids using well known techniques to inactivate the receptor binding and/or functional activity of the scFv.

The term “identity” or “identical” as used herein refers to the percentage number of amino acids that are identical or constitute conservative substitutions. Homology may be determined using sequence comparison programs such as GAP (Deveraux et al., 1984, Nucleic Acids Research 12, 387-395), which is incorporated herein by reference. In this way sequences of a similar or substantially different length to those cited herein could be compared by insertion of gaps into the alignment, such gaps being determined, for example, by the comparison algorithm used by GAP.

As used herein, the terms “disease”, “disorder” or “condition” refers to a disruption of or interference with normal function, and is not to be limited to any specific condition, and will include diseases or disorders.

As used herein, a subject “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a protein described herein to thereby reduce or eliminate at least one symptom of a specified disease or condition or to slow progression of the disease or condition.

As used herein, the terms “preventing”, “prevent” or “prevention” include administering a protein of the disclosure to thereby stop or hinder the development of at least one symptom of a condition or disease. This term also encompasses treatment of a subject in remission to prevent or hinder relapse. For example, a subject suffering from relapsing-remitting multiple sclerosis is treated during remission to thereby prevent a relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change in a factor associated with a disease or condition as hereinbefore described. For example, the effective amount may be sufficient to effect a change in the level of platelets. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of binding proteins. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the antibody or antigen binding fragment thereof are outweighed by the therapeutically beneficial effects. In one example, a therapeutically effective amount shall be taken to mean a sufficient quantity of binding protein to reduce or inhibit one or more symptoms of an inflammatory neurological disease (e.g., MS) or a complication thereof.

As used herein, the term “prophylactically effective amount” shall be taken to mean a sufficient quantity of membrane targeted binding protein to prevent or inhibit or delay the onset of one or more detectable symptoms of an inflammatory neurological disease (e.g., MS) or a complication thereof.

As used herein, the term “subject” shall be taken to mean any animal including humans, for example a mammal. Exemplary subjects include but are not limited to humans and non-human primates. For example, the subject is a human.

### Binding Proteins

The present disclosure provides a binding protein comprising an extracellular domain of CD39. The present disclosure further provides a binding protein comprising an extracellular domain of CD39 and a binding region that specifically binds GPIIb/IIIa. In one example, a binding protein of the present disclosure comprises a sequence at least about 85% or 90% or 95% or 97% or 98% or 99% identical to a sequence disclosed herein.

### Extracellular Domain of CD39

The present disclosure provides a binding protein comprising an extracellular domain of CD39.

CD39 is a cell-surface antigen that was originally identified as a marker for mature B cells, but is also expressed on less mature B cells, Epstein-Barr Virus-transformed B cells, activated T cells, endothelial cells and some myeloid cell lines. CD39 is an ecto-ADPase (apyrase) responsible for the maintenance of blood fluidity, thus maintaining platelets in the baseline (resting) state. This is accomplished by metabolism of adenosine triphosphate (ATP) to the major platelet agonist, adenosine diphosphate (ADP), and to adenosine monophosphate.

As used herein, the term “extracellular domain of CD39” includes naturally occurring CD39, but also variants thereof, e.g., fragments or sequence variants where one or more residues have been inserted, deleted or substituted, retaining the biological activity of naturally occurring CD39.

In one example, the extracellular domain of CD39 is human derived. For example, the extracellular domain of CD39 comprises a sequence set forth in SEQ ID NO: 4.

In another example, the extracellular domain of CD39 is recombinant.

### GPIIb IIIa Binding Region

A binding protein of the present disclosure comprises a binding region that is an inhibitor of GPIIb/IIIa receptor function and/or activity.

In one example, the binding region specifically binds an epitope on GPIIb/IIIa recognised by a scFv consisting of a sequence set forth in SEQ ID NO: 1.

In one example, the binding region competitively inhibits binding of a scFv consisting of a sequence set forth in SEQ ID NO: 1 to an epitope on GPIIb/IIIa.

In one example, the binding region comprises an antibody variable region, e.g., is an antibody or an antibody fragment that binds to GPIIb/IIIa. For example, the antibody variable region binds specifically to activated GPIIb/IIIa.

Suitable antibodies and proteins comprising variable regions thereof are known in the art and/or described herein.

In one example, the binding protein comprises a binding region, wherein the binding region is a protein comprising a Fv. For example, the protein comprises a single chain Fv fragment (scFv).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). In one example, the linker comprises the sequence SSGS.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Other Antibodies or Antigen Binding Fragments

Exemplary antibodies or antigen binding fragments thereof for use in the present disclosure are described herein or known in the art and include:
a humanized antibody or fragment thereof, e.g., a protein comprising a human-like variable region, which includes CDRs from an antibody from a non-human species (e.g., mouse or rat or non-human primate) grafted onto or inserted into FRs from a human antibody (e.g., produced by methods described in U.S. Pat. Nos. 5,225,539, 6,054,297, 7,566,771 or U.S. Pat. No. 5,585,089)
a human antibody or fragment thereof, e.g., antibodies having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (e.g., produced by methods described in U.S. Pat. No. 5,565,332) and affinity matured forms of such antibodies.
a synhumanized antibody or fragment thereof, e.g., an antibody that includes a variable region comprising FRs from a New World primate antibody variable region and CDRs from a non-New World primate antibody variable region (e.g., produced by methods described in WO2007019620).
a primatized antibody or fragment thereof, e.g., an antibody comprising variable region(s) from an antibody generated following immunization of a non-human primate (e.g., a cynomolgus macaque) (e.g., produced by methods described in U.S. Pat. No. 6,113,898).
a chimeric antibody or chimeric antigen binding fragment, e.g., an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass (e.g., produced by methods described in U.S. Pat. Nos. 6,331,415; 5,807,715; 4,816,567 and 4,816,397).
a deimmunized antibody or antigen binding fragment thereof, e.g., antibodies and fragments that have one or more epitopes, e.g., B cell epitopes or T cell epitopes removed (i.e., mutated) to thereby reduce the likelihood that a subject will raise an immune response against the antibody or protein (e.g., as described in WO2000034317 and WO2004108158).
a bispecific antibody or fragment thereof, e.g., an antibody comprising two types of antibodies or antibody fragments (e.g., two half antibodies) having specificities for different antigens or epitopes (e.g., as described in U.S. Pat. No. 5,731,168).

Additional exemplary antibody fragments for use in the present disclosure are described herein or known in the art and include:
single-domain antibodies (domain antibody or dAb), e.g., a single polypeptide chain comprising all or a portion of the heavy chain variable domain of an antibody.
a diabody, triabody, tetrabody or higher order protein complex (e.g., as described in WO98/044001 and/or WO94/007921).
a half-antibody or a half-molecule, e.g., a protein comprising a single heavy chain and a single light chain.

The present disclosure also contemplates other antibodies and antibody fragments, such as:
minibodies, e.g., as described in U.S. Pat. No. 5,837,821;
heteroconjugate proteins, e.g., as described in U.S. Pat. No. 4,676,980;
heteroconjugate proteins produced using a chemical cross-linker, e.g., as described in U.S. Pat. No. 4,676,980; and
Fab₃ (e.g., as described in EP19930302894).

### Linkers

The present disclosure provides a binding protein comprising an extracellular domain of CD39 and a binding region which specifically binds to GPIIb/IIIa.

In one example, the extracellular domain of CD39 is conjugated to the binding region. The extracellular domain of CD39 can be directly or indirectly bound to the binding region (e.g., can comprise a linker in the case of indirect binding). For example, the extracellular domain of CD39 and the binding region are covalently linked by an amide bond. The present disclosure encompasses other forms of covalent and non-covalent linkages. For example, the regions can be linked by a chemical linker.

In one example, the linker is a flexible linker, e.g., a flexible peptide linker. For example, the extracellular domain of CD39 is linked to the binding region via a flexible linker.

In one example, the linker is a peptide linker.

In one example, the extracellular domain of CD39 is linked to the binding region via a linker. For example, the linker is a linker peptide. For example, the extracellular domain of CD39 is linked to the binding region via a linker wherein the linker is a peptide linker comprising between 3 and 30 amino acids in length. For example, the linker sequence is about 3 amino acids in length. In one example, the linker comprises the sequence (Ala)_{3.}

In one example, an intervening peptidic linker may be introduced between the extracellular domain of CD39 and the binding region.

In one example, the linker is a flexible linker. For example, the linker joins the extracellular domain of CD39 to the N- or C-terminus of a heavy chain or domain thereof or a light chain or domain thereof of the binding region (i.e., which is a scFv).

A “flexible” linker is an amino acid sequence which does not have a fixed structure (secondary or tertiary structure) in solution. Such a flexible linker is therefore free to adopt a variety of conformations. Flexible linkers suitable for use in the present disclosure are known in the art. Flexible linkers are also disclosed in WO1999045132.

The linker may comprise any amino acid sequence that does not substantially hinder interaction of the binding region with its target. Preferred amino acid residues for flexible linker sequences include, but are not limited to, glycine, alanine, serine, threonine proline, lysine, arginine, glutamine and glutamic acid.

The linker sequences between the binding regions preferably comprise five or more amino acid residues. The flexible linker sequences according to the present disclosure consist of 3 or more residues, preferably, 3, 4, 5, 6, 7, 8, 9, 10, 11, 12, 13, 14, 15, 16, 17, 18, 19 or 20 or more residues. In a highly preferred embodiment of the invention, the flexible linker sequences consist of 3, 5, 7, 10 or 16 residues.

In one example, the linker is a rigid linker. A “rigid linker” (including a “semi-rigid linker”) refers to a linker having limited flexibility. For example, the relatively rigid linker comprises the sequence (EAAAK)ₙ, where n is between 1 and 3. The value of n can be between 1 and about 10 or between about 1 and 100. For example, n is at least 1, or at least 2, or at least 3, or at least 4, or at least 5, or at least 6, or at least 7, or at least 8, or at least 9, or at least 10. In one example, n is less than 100. For example, n is less than 90, or less than about 80, or less than about 60, or less than about 50, or less than about 40, or less than about 30, or less than about 20, or less than about 10. A rigid linker need not completely lack flexibility.

In one example, the linker is a cleavable linker. For example, the linker comprises a cleavage site for a peptidase. For example, the linker comprises a cleavage site for urokinase, pro-urokinase, plasmin, plasminogen, TGFβ, staphylokinase, Thrombin, a coagulation factor (e.g., Factor IXa, Factor Xa) or a metalloproteinase, such as an interstitial collagenase, a gelatinase or a stromelysin. Exemplary cleavable linkers are described in U.S. Pat. Nos. 6,004,555, 5,877,289, 6,093,399 and 5,877,289.

### Treating or Preventing an Inflammatory Neurological Disease

As discussed herein, the present disclosure provides a method of treating or preventing an inflammatory neurological disease in a subject, the method comprising administering the binding protein of the present disclosure or the composition of the present disclosure to a subject in need thereof. In one example, the present disclosure provides a method of treating an inflammatory neurological disease in a subject in need thereof.

The present disclosure also provides for use of a binding protein of the present disclosure for treating or preventing an inflammatory neurological disease in a subject comprising administering the binding protein of the present disclosure or the composition of the present disclosure to a subject in need thereof. In one example, the present disclosure provides for use of a binding protein of the present disclosure for treating an inflammatory neurological disease in a subject in need thereof.

In one example, the inflammatory neurological disease is selected from the group consisting of multiple sclerosis, systemic lupus erythematosus (SLE), Guillain-Barre syndrome, Lambert-Eaton myasthenic syndrome, myasthenia gravis, transverse myelitis, leukodystrophy and progressive multifocal leukoencephalopathy.

In one example, the inflammatory neurological disease is MS.

In one example, the inflammatory neurological disease is systemic lupus erythematosus (SLE).

In one example, the inflammatory neurological disease is Guillain-Barre syndrome.

In one example, the inflammatory neurological disease is Lambert-Eaton myasthenic syndrome.

In one example, the inflammatory neurological disease is myasthenia gravis.

In one example, the inflammatory neurological disease is transverse myelitis.

In one example, the inflammatory neurological disease is leukodystrophy.

In one example, the inflammatory neurological disease is progressive multifocal leukoencephalopathy.

In one example, the subject suffers from MS. For example, the subject has been diagnosed as having MS. Four disease courses have been identified in MS which will be apparent to the skilled person: clinically isolated syndrome (CIS), relapsing-remitting MS (RMS), primary progressive MS (PPMS) and secondary progressive MS (SPMS). In one example, the subject suffers from clinically isolated syndrome (CIS). In another example, the subject suffers from relapsing-remitting MS (RMS). In a further example, the subject suffers from primary progressive MS (PPMS). In another example, the subject suffers from secondary progressive MS (SPMS).

Methods of diagnosing a subject with MS (including the disease course) are known in the art and/or described herein, including neurological examination, magnetic resonance imaging (MRI), visual evoked potentials (VEP) and cerebrospinal fluid analysis. In one example, a subject is diagnosed with MS according to the Revised McDonald Criteria (published 2017) by the International Panel on the Diagnosis of Multiple Sclerosis. For example, the subject is diagnosed according to the criteria set out in Table 1.

TABLE 1
Revised McDonald Criteria for the diagnosis of MS (2017)

| | |
|---|---|
| Clinical presentation | Additional criteria to make MS diagnosis |

. . . in a person who has experienced a typical attack/CIS at onset

| | |
|---|---|
| 2 or more attacks and | None. DIS and DIT have been met. |
| clinical evidence of 2 or | |
| more lesions; OR | |
| 2 or more attacks and | |
| clinical evidence of 1 | |
| lesion with clear | |
| historical evidence of | |
| prior attack involving | |
| lesion in different | |
| location | |
| 2 or more attacks and | DIS shown by one of these criteria: |
| clinical evidence of 1 | additional clinical attack implicating |
| lesion | different CNS site |
| | 1 or more MS-typical T2 lesions in 2 |
| | or more areas of CNS: periventricular, |
| | cortical, juxtacortical, infratentorial or |
| | spinal cord |
| 1 attack and clinical | DIT shown by one of these criteria: |
| evidence of 2 or more | Additional clinical attack |
| lesions | Simultaneous presence of both enhancing |
| | and non-enhancing MS-typical MRI |
| | lesions, or new T2 or enhancing MRI lesion |
| | compared to baseline scan (without regard |
| | to timing of baseline scan) |
| | CSF oligoclonal bands |
| 1 attack and clinical | DIS shown by one of these criteria: |
| evidence of 1 lesion | Additional attack implicating different CNS |
| | site |
| | 1 or more MS-typical T2 lesions in 2 or |
| | more areas of CNS: periventricular, |
| | cortical, juxtacortical, infratentorial or |
| | spinal cord |
| | AND |
| | DIT shown by one of these criteria: |
| | additional clinical attack |
| | Simultaneous presence of both enhancing |
| | and non-enhancing MS-typical MRI |
| | lesions, or new T2 or enhancing MRI lesion |
| | compared to baseline scan (without regard |
| | to timing of baseline scan) |
| | CSF oligoclonal bands |

. . . in a person who has steady progression of disease since onset

| | |
|---|---|
| 1 year of disease | DIS shown by at least two of these criteria: |
| progression (retrospective | 1 or more MS-typical T2 lesions |
| or prospective) | (periventricular, cortical, juxtacortical or |
| | infratentorial) |
| | 2 or more T2 spinal cord lesions |
| | CSF oligoclonal bands |

DIT = Dissemination in time
CNS = central nervous system
CSF = cerebrospinal fluid
DIS = Dissemination in space
T2 lesion = hyperintense lesion on T2-weighted MRI

In one example, the subject suffers from a symptom of MS. For example, a subject is suffering from a symptom of MS, such as:
Motor control, including, for example, muscular spasms and problems with weakness, coordination, balance and functioning of the arms and legs;
Fatigue, including heat sensitivity;
Other neurological symptoms, including, for example, vertigo, pins and needles, neuralgia and visual disturbances;
Continence problems, including bladder incontinence and constipation; and/or
Neuropsychological symptoms, including, for example, memory loss, depression and cognitive difficulties.

In one example, the subject is at risk of developing MS. A subject is at risk if he or she has a higher risk of developing MS than a control population. The control population may include one or more subjects selected at random from the general population (e.g., matched by age, gender, race and/or ethnicity) who have not suffered from or have a family history of MS or other inflammatory neurological disease. A subject can be considered at risk for MS if a “risk factor” associated with MS is found to be associated with that subject. A risk factor can include any activity, trait, event or property associated with a given disorder, for example, through statistical or epidemiological studies on a population of subjects. A subject can thus be classified as being at risk for MS even if studies identifying the underlying risk factors did not include the subject specifically.

In one example, a method of the disclosure reduces any symptom of MS known in the art or described herein.

As will be apparent to the skilled person a “reduction” in a symptom of MS in a subject will be comparative to another subject who has also suffered from MS but who has not received treatment with a method described herein or to the subject prior to treatment. This does not necessarily require a side-by-side comparison of two subjects. Rather population data can be relied upon. For example a population of subjects suffering from MS who have not received treatment with a method described herein (optionally, a population of similar subjects to the treated subject, e.g., age, weight, etc) are assessed and the mean values are compared to results of a subject or population of subjects treated with a method described herein.

A method of the present disclosure may also include co-administration of the at least one binding protein according to the disclosure together with the administration of another therapeutically effective agent for the prevention or treatment of an inflammatory neurological disease (e.g., MS) and/or a symptom thereof.

In one example, the binding protein of the disclosure is used in combination with at least one additional known compound or therapeutic which is currently being used or is in development for preventing or treating an inflammatory neurological disease (e.g., MS) and/or a symptom thereof. Compounds currently used in the treatment of MS are known in the art and/or described herein. For example, the compound or therapeutic is selected from the group consisting of ocrelizumb (e.g., Ocrevus®), β-interferon (e.g., Betaseron®, Avonex® and Rebif®), Copaxone® (copolymer-1; glatiramer acetate), mitoxantrone (e.g., Novantrone®), and natalizumab (e.g., Tysabri®).

As will be apparent from the foregoing, the present disclosure provides methods of concomitant therapeutic treatment of a subject, comprising administering to a subject in need thereof an effective amount of a first agent and a second agent, wherein the first agent is a binding protein of the present disclosure, and the second agent is also for the prevention or treatment of MS and/or a symptom thereof.

As used herein, the term “concomitant” as in the phrase “concomitant therapeutic treatment” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agent, wherein the second or additional agent, for example, may have been previously administered. A concomitant therapeutic treatment method may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g. a human).

The optimum concentration of the active ingredient(s) in the chosen medium can be determined empirically, according to procedures known to the skilled artisan, and will depend on the ultimate pharmaceutical formulation desired.

The dosage ranges for the administration of the binding protein of the disclosure are those large enough to produce the desired effect. For example, the composition comprises an effective amount of the binding protein. In one example, the composition comprises a therapeutically effective amount of the binding protein. In another example, the composition comprises a prophylactically effective amount of the binding protein.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the binding protein is administered at an initial (or loading) dose which is higher than subsequent (maintenance doses). For example, the binding protein is administered at an initial dose of between about 10 mg/kg to about 30 mg/kg. The binding protein is then administered at a maintenance dose of between about 0.0001 mg/kg to about 10 mg/kg. The maintenance doses may be administered every 7-35 days, such as, every 7 or 14 or 28 days.

In some examples, a dose escalation regime is used, in which a binding protein is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the binding protein, by being given more than one exposure or set of doses, such as at least about two exposures of the binding protein, for example, from about 2 to 60 exposures, and more particularly about 2 to 40 exposures, most particularly, about 2 to 20 exposures.

In one example, any retreatment may be given when signs or symptoms of disease return, e.g., a neurological episode.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of a binding protein according to the methods of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses, e.g., either during or after development of a condition.

### Screening Assays

Binding proteins of the present disclosure are readily screened for biological activity, for example, as described below.

### Expression Assays

A binding protein of the present disclosure that reduces or inhibits GPIIb/IIIa receptor function and/or activity is identified by contacting platelets with the binding protein and determining the level of expression of platelet markers, e.g., CD41, PF4 and/or CXCL4.

Suitable methods for determining gene expression at the nucleic acid level are known in the art and include, for example, quantitative polymerase chain reaction (qPCR) or microarray assays. Suitable methods for determining expression at the protein level are also known in the art and include, for example, enzyme-linked immunosorbent assay (ELISA), fluorescence linked immunosorbent assay (FLISA), immunofluorescence, Western blotting, or flow cytometry.

In one example, the neuro-inflammatory parameters of EAE are assessed. For example, antibodies against astrocytic glial fibrillary acidic protein (GFAP), microglial ionized calcium-binding adapter protein 1 (Ibal) and myelin basic protein (MBP) are used to assess glial reactivity and demyelination.

### Measuring Activity

Proteins of the present disclosure can also be assayed to test for CD39 activity. In one example, the CD39 activity is assayed in vitro. For example, activity, cell suspensions are incubated with 50 μmolar 14C-labeled ADP or ATP in 50 μl assay buffer for five minutes and the reaction terminated by addition of a stop solution (160 mM EDTA (pH 7) and 17 mM ADP in 0.9% saline). Suspended cells are then removed by centrifugation and the supernatant decanted for analysis of the reaction products. In one example, thin-layer chromatography (TLC) is used to separate labeled nucleotides, nucleosides, and bases. Radioactivity is measured to assess metabolism of 14C-labeled ADP or ATP by CD39. In one example, data is expressed as a percentage of ADP or ATP metabolized or as picomoles nucleotide metabolized per minute per 50,000 or 100,000 cells.

### In Vivo Assays

Binding proteins of the present disclosure can also be assessed for therapeutic efficacy in an animal model of a condition, for example, an inflammatory neurological disease.

For example, the binding protein is administered to a model of multiple sclerosis, for example, EAE models in which a mouse or rat is immunized with a myelin sheath protein or peptide derived therefrom (e.g., MOG, MBP or PLP) and an immune response is generated against the protein thereby inducing a model of multiple sclerosis. Exemplary EAE models are reviewed in, for example Tsunoda and Fujinami, J. Neuropathol. Exp. Neurol. 55: 673-686, 1996.

### Competitive Binding Assays

Assays for determining a binding protein that competitively inhibits binding of a scFv of the disclosure will be apparent to the skilled artisan. For example, the binding protein of the disclosure is conjugated to a detectable label, for example, a fluorescent label or a radioactive label. The labeled protein and the test binding protein are then mixed and contacted with GPIIb/IIIa or a peptide comprising an epitope thereof. The level of labeled protein is then determined and compared to the level determined when the labeled protein is contacted with the GPIIb/IIIa or the peptide comprising an epitope thereof in the absence of the binding protein. If the level of labeled protein is reduced in the presence of the binding protein compared to the absence of the binding protein, the binding protein competitively inhibits binding of the scFv.

### Epitope Mapping Assays

In another example, the epitope bound by a protein described herein is mapped. Epitope mapping methods will be apparent to the skilled artisan. For example, a series of overlapping peptides spanning the GPIIb/IIIa sequence or a region thereof comprising an epitope of interest, for example, peptides comprising 10 to 15 amino acids are produced. The binding protein is then contacted to each peptide or a combination thereof and the peptide(s) to which it binds determined. This permits determination of peptide(s) comprising the epitope to which the binding protein binds. If multiple non-contiguous peptides are bound by the protein, the protein may bind a conformational epitope.

Alternatively, or in addition, amino acid residues within GPIIb/IIIa are mutated, for example, by alanine scanning mutagenesis, and mutations that reduce or prevent protein binding are determined. Any mutation that reduces or prevents binding of the binding protein is likely to be within the epitope bound by the protein.

### Pharmaceutical Compositions

Suitably, in compositions or methods for administration of the binding protein of the disclosure to a subject, the binding protein is combined with a pharmaceutically acceptable carrier as is understood in the art. Accordingly, one example of the present disclosure provides a composition (e.g., a pharmaceutical composition) comprising the binding protein of the disclosure combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A binding protein of the present disclosure is useful for parenteral, topical, oral, or local administration, aerosol administration, or transdermal administration, for prophylactic or for therapeutic treatment. In one example, the binding protein is administered parenterally, such as subcutaneously or intravenously. For example, the binding protein administered intravenously.

Formulation of a binding protein to be administered will vary according to the route of administration and formulation (e.g., solution, emulsion, capsule) selected. An appropriate pharmaceutical composition comprising a binding protein to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The membrane targeted binding protein can be stored in the liquid stage or can be lyophilized for storage and reconstituted in a suitable carrier prior to use according to art-known lyophilization and reconstitution techniques.

### Kits and Other Compositions of Matter

Another example of the disclosure provides kits containing a binding protein of the present disclosure useful for the treatment or prevention of an inflammatory neurological disease as described above.

In one example, the kit comprises (a) a container comprising a binding protein optionally in a pharmaceutically acceptable carrier or diluent; and (b) a package insert with instructions for treating or preventing an inflammatory neurological disease (e.g., MS) in a subject.

In one example, the kit comprises (a) at least one binding protein; (b) instructions for using the kit in treating or preventing the inflammatory neurological disease in the subject; and (c) optionally, at least one further therapeutically active compound or drug.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating an inflammatory neurological disease (e.g., MS) and may have a sterile access port (for example, the container may be an intravenous solution bag or a vial having a stopper pierceable by a hypodermic injection needle). At least one active agent in the composition is the binding protein. The label or package insert indicates that the composition is used for treating a subject eligible for treatment, e.g., one having or predisposed to developing an inflammatory neurological disease, with specific guidance regarding dosing amounts and intervals of binding protein and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprises a second medicament, wherein the binding protein is a first medicament, and which article further comprises instructions on the package insert for treating the subject with the second medicament, in an effective amount. The second medicament may be a therapeutic protein set forth above.

The present disclosure includes the following non-limiting Examples.

### EXAMPLES

### Example 1: Platelet Accumulation Begins During the Pre-Clinical Stage of EAE

An experimental autoimmune encephalomyelitis (EAE) model of multiple sclerosis (MS) was used to determine the relationship between platelets and EAE development. Experimentation was based on the MOG₃₅₋₅₅-induced C57Bl/6 variant (as previously described in Pham et al., 2011). Briefly, EAE was induced in female C57Bl/6 mice (9-12 weeks of age) using a MOG₃₅₋₅₅ peptide and disease progression was monitored on a daily basis, as previously described (Dang et al., 2015). Control groups included normal and vehicle-only mice, sex and age matched. Mice were randomly assigned to each group.

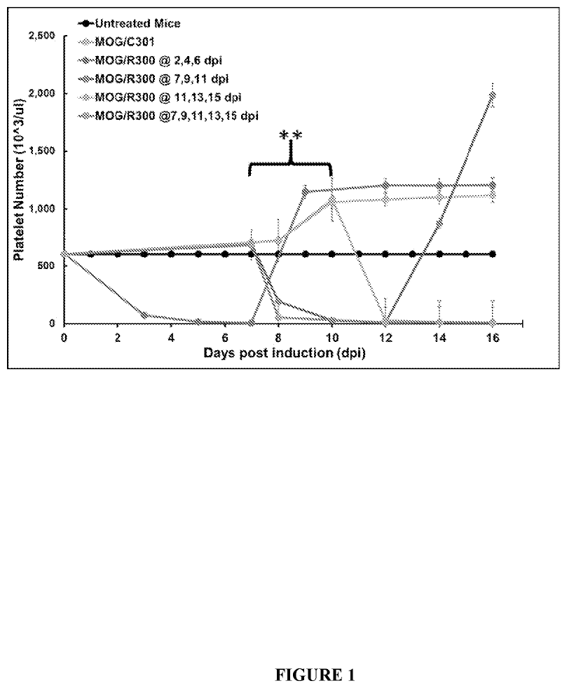

Platelet counts in blood were performed over the disease course, from pre-clinical stage to a score of 3 (FIG. 1). Blood samples collected via sub-mandibular bleeds were estimated on an automated hematology analyzer in MOG₃₅₋₅₅-induced/C301-treated, as well as all MOG₃₅₋₅₅-induced/R300 treated groups. In all R300-treated groups, a 98% reduction in platelets was seen within 24 hours. In MOG₃₅₋₅₅-induced/C301-treated group a gradual rise in platelet numbers from 0 to 7 days post injection (dpi) was observed, followed by a sudden and significant increase at 7-8 dpi before reaching a plateau at 9 dpi, which was maintained until experimental end point.

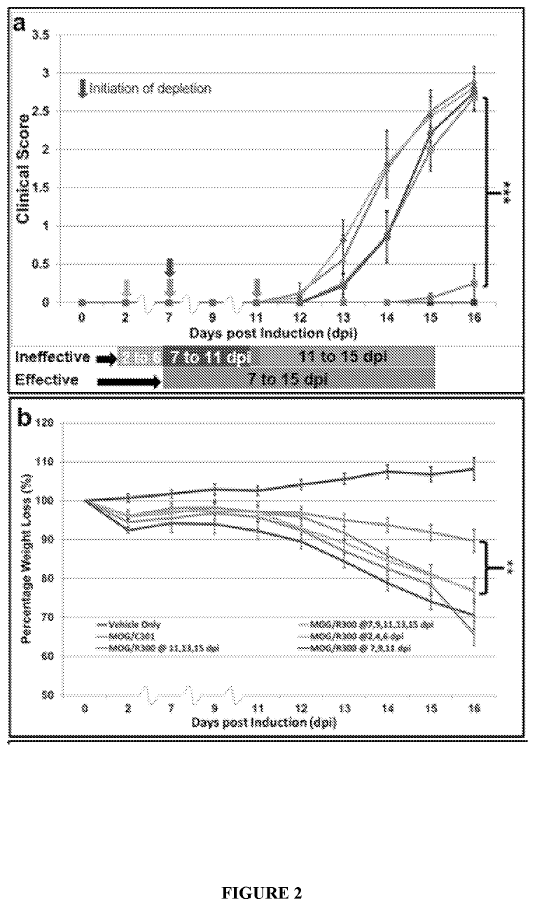

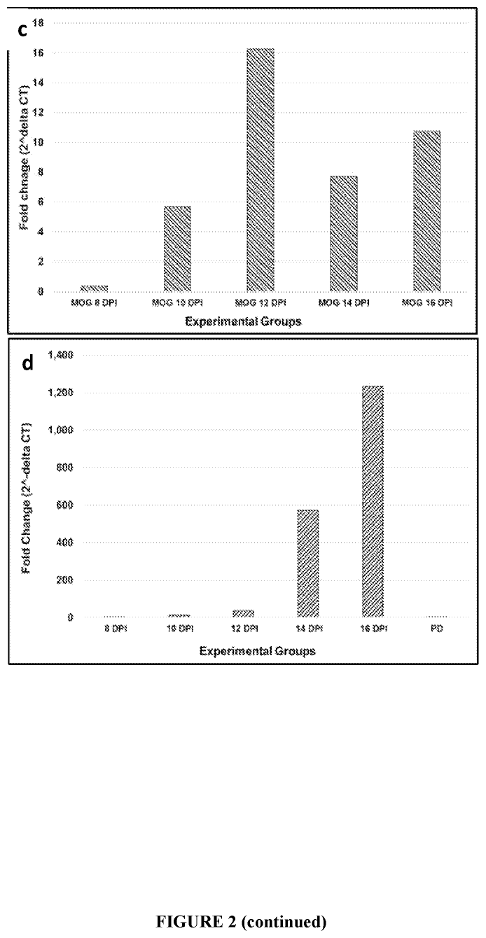

These data identified the timing of platelet accumulation ahead of that of clinical onset, which occurs at 13 to 14 dpi in this EAE model (FIG. 2a).

### Example 2: Platelet Depletion Immediately Prior to T Cell Accumulation Eliminates EAE

To investigate whether platelet accumulation is key to disease development, platelet depletion was performed as previously described (Nieswandt et al., 2000). Briefly, platelet depletion was achieved by intravenous injection of platelet depletion antibody cocktail, containing a rat monoclonal antibody against CD42b (R300, EMFRET Analytics GMBH & Co KG, Eibelstadt, Germany) at a dose of 0.5 mg/kg body weight. A non-immune rat Ig preparation (C301, Emfret Analytics) was administered at the same concentration, as control. Sub-mandibular bleeds were carried out 24 hours after each injection of the R300 or C301 preparations, for estimations of platelet numbers using an automated haematology analyser, Sysmex XS-1000i (Sysmex America Inc., Mundelein, IL, USA).

Treatment regimens varied in initiation time and treatment duration as shown in Table 1 below and the R300 preparation was administered over the 2-6, 7-11, or 11-15 dpi periods. In all cases, platelet depletion had no significant effect on the timing of clinical disease onset, or on mean clinical scores at experimental end-point, nor on disease duration, relative to the MOG₃₅₋₅₅-induced/C301-treated group (FIG. 2 and Table 2).

The only significantly different measure was that of cumulative disease scores in the 2-6 dpi and 11-15 dpi depletion groups relative to the MOG₃₅₋₅₅-induced/C301-treated group (p<0.05) (Table 2). Irrespective of treatment regimen, platelet depletion with R300 was >98% (Table 2) and platelet numbers recovered rapidly within 48 hours of cessation of treatment, exceeding those of the MOG₃₅₋₅₅-induced/C301-treated group (FIG. 1). Strikingly, when platelet depletion was initiated at 7 dpi, but sustained until 15 dpi, clinical EAE was essentially abolished (p<0.0000015, FIG. 2a, Table 2) and percentage weight loss (FIG. 2b) significantly reduced (p<0.01), compared with the MOG₃₅₋₅₅-induced/C301-treated group.

H&E staining of spinal cord tissues at experimental end point were also performed. No perivascular inflammation in the 7-15 dpi MOG₃₅₋₅₅-induced/R300-treated group was observed. However, severe lesions in the MOG₃₅₋₅₅/R300-treated groups with short-term depletion initiated at 2, 7 or 11 dpi, or other MOG-induced control groups was observed.

Immunochemical evaluation of other parameters of neuroinflammation in the same tissues was also performed, namely astrocytic and microglial glial reactivity (identified by glial fibrillary acidic protein [GFAP] and ionized calcium binding adaptor protein 1 [Ibal] respectively), as well as demyelination (identified by myelin basic protein [MBP] staining). Both glial reactivity and demyelination were greatly reduced in the MOG₃₅₋₅₅-induced/R300 treated group where depletion was initiated at 7 dpi and maintained. These parameters were found to be elevated in experimental groups exposed to short-term platelet depletion (i.e., the MOG₃₅₋₅₅-induced/C301-treated group) and were very similar to levels seen in the vehicle-only group. These results demonstrate that platelet depletion is highly effective when initiated at 7 dpi, namely prior to clinical onset and corresponding to the timing of significant rise in circulating platelet numbers (FIG. 1). However, sustained depletion is required to completely eliminate EAE development.

TABLE 2
Treatment regimens

| | | | | | | | | | | Mean |
|---|---|---|---|---|---|---|---|---|---|---|
| | | | | | | | | | Mean | percent |
| | | | | | Mean | Mean | Mean | Mean | platelet | weight |
| | Start of | End of | No. of | | disease | clinical | disease | cumulative | number at | change at |
| | deple- | deple- | injec- | Anti- | onset | score at | duration | clinical | endpoint | endpoint vs |
| Group | tion | tion | tions | body | (dpi) | endpoint | (days) | score | (×10³/μl) | normal |
| Normal | N/A | N/A | N/A | N/A | N/A | N/A | N/A | N/A | 606 ± 70 | N/A |
| Vehicle | 7 dpi | 15 dpi | 5 | R300 | N/A | N/A | N/A | N/A | 13 ± 3 | 108.1 ± 2.0 |
| ctrl | | | | | | | | | | |
| Vehicle | 7 dpi | 15 dpi | 5 | C301 | N/A | N/A | N/A | N/A | 780 ± 55 | 114.8 ± 4.6 |
| ctrl | | | | | | | | | | |
| MGO- | N/A | N/A | N/A | N/A | 13 ± 0.4 | 2.9 ± 0.15 | 3.1 ± 0.4 | 7.4 ± 1.00 | 1483 ± 135 | 75.0 ± 1.5 |
| induced | | | | | | | | | | |
| MGO- | 7 dpi | 15 dpi | 5 | C301 | 14 ± 0.3 | 2.7 ± 0.16 | 3.0 ± 0.3 | 5.8 ± 0.82 | 1115 ± 155 | 76.7 ± 2.2 *** |
| induced | | | | | | | | | | |
| MGO- | 2 dpi | 6 dpi | 3 | R300 | 13 ± 0.4 | 2.8 ± 0.10 | 3.6 ± 0.4 | 8.6 ± 0.68* | 1204 ± 222 | 77.0 ± 2.9 *** |
| induced | | | | | | | | | | |
| MGO- | 11 dpi | 15 dpi | 3 | R300 | 14 ± 0.3 | 2.9 ± 0.19 | 3.5 ± 0.3 | 8.3 ± 0.84* | 11 ± 2 | 65.9 ± 3.1*** |
| induced | | | | | | | | | | |
| MGO- | 7 dpi | 11 dpi | 3 | R300 | 14 ± 0.5 | 2.8 ± 0.3 | 2.3 ± 0.4 | 4.6 ± 0.88 | 1985 ± 474 | 70.6 ± 3.5*** |
| induced | | | | | | | | | | |
| MGO- | 7 dpi | 15 dpi | 5 | R300 | 15# | 0.25 ± | 0.3 ± 0.3 | 0.3 ± 0.63 | 11 ± 4 | 89.7 ± |
| induced | | | | | | 0.25*** | | | | 2.9***, ** |

Error bars represent SEM and significance level * = p ≤ 0.05, ** = p ≤ 0.01 and *** = p ≤ 0.001. NA = Not applicable; # represents a single mouse from this group which exhibited a score of 2 at 16 dpi.

### Example 3: Platelets Infiltrate the Parenchyma Ahead of Lymphocytic Infiltration

The parenchymal accumulation of platelets and T cells was also investigated. Briefly, qPCR analyses of the platelet marker CD41 and the T cell marker CD3 over the disease course, using cDNA generated from PBS-perfused spinal cord tissue RNA was performed. Tissues were sampled from three treatment regimens, namely, (a) MOG₃₅₋₅₅-induced/no further treatment from (5 time points, between 8 and 16 dpi), (b) MOG₃₅₋₅₅-induced/R300-treated over the 7-15 dpi period (experimental end-point only) and (c) vehicle-only/R300-treated (end-point).

Results showed rapid parenchymal accumulation of CD41 only in the MOG₃₅₋₅₅-induced/no further treatment group, beginning between 8-10 dpi and reaching maximum levels by 12 dpi, but decreasing to intermediate levels by experimental end-point (FIG. 2c). CD3 accumulation was first observed in the same group between 10-12 dpi, and increased until 16 dpi (FIG. 2d). Comparatively, CD3 accumulation was almost undetectable in cDNA isolated from platelet-depleted animals. No comparable changes in platelet, or CD3 markers, were found in control groups. The peak of parenchymal platelet invasion preceded that of CD3 cells and platelet depletion was associated with absence of CD3 accumulation.

These data suggest that platelet accumulation drives CD3 cell recruitment into the parenchyma, rather than the reverse and provide an explanation for the efficacy of platelet depletion when initiated from 7 dpi rather than clinical onset.

### Example 4: Platelets Infiltrate Both Grey and White Matter CNS

The presence of platelets in the parenchyma was confirmed by immunochemistry with combined anti-CD41 and anti-CD42b. Confirmation that elements stained were platelets was provided by coincidence of staining, small size and absence of nuclei in elements stained. The small size of platelets precludes their early detection at time points identified by expression analysis; however, the timing of their earliest detection in the parenchyma differed between white and grey matter. In white matter, platelets were found by 12 dpi, generally in association with perivascular lesions and reaching high levels by 16 dpi. Comparatively, in grey matter platelets were clearly detectable by 10 dpi and commonly observed in close association with neuronal cell bodies. Double immunochemistry against serum albumin (as a marker of compromised blood brain barrier) and platelets showed that at 10 dpi platelet infiltration in grey matter was coincident with severe albumin leakage but not inflammatory infiltration, as evidenced by the absence of perivascular cuffs. Platelets were not observed in either the white or the grey matter in the 7-15 dpi MOG₃₅₋₅₅-induced/R300-treated group. These data confirm that platelet invasion was associated with early compromised blood brain barrier, but ahead of inflammatory cells, in the grey matter compartment.

To further address the significance of the early entry of platelets in grey matter parenchyma, immunochemical analysis of platelets and the platelet-specific product platelet factor 4 (PF4, or CXCL4) was performed. PF4 is released during platelet activation and exhibits a wide range of pro-inflammatory activities. Platelet and PF4 co-localization was evident reaching a peak by 12 dpi, namely at the time corresponding to the peak of platelet accumulation as shown in FIG. 2c.

### Example 5: Platelets Infiltrate the Retina from the Pre-Clinical Stage without Lymphocytic Infiltration

Optic neuritis is an early symptom in EAE, similarly to MS. Therefore, to further demonstrate the pivotal role of platelets in neuroinflammation, platelet infiltration and accumulation in the retina from pre-clinical disease stage was examined.

In retinal flatmounts, CD42b/CD31 immunochemistry detected the presence of platelets in the vasculature of both sham-injected and EAE-induced mice. However, whilst platelets remained confined to blood vessels in sham-injected mice, these elements were clearly seen to leak from blood vessels as early as 9 dpi and throughout the disease course. Significantly, platelet leakage was associated with the inner retinal layer only, as shown in coronal sections of the eyeball, which supports the notion that platelet leakage is a specific process.

In the retina, platelet accumulation was not followed by that of CD3 cells. CD42b/CD3 immunochemistry showed that at 9 dpi no CD3 positive cells had invaded the retina, whilst CD42b positive elements were already abundant. By 14 dpi, CD42b-positive immunostaining had increased, however, CD3 cells remained undetectable. This was in contrast with the optic nerve where platelets could be identified from 9 dpi onwards, but not CD3 cells, while both platelets and abundant CD3 cells were present by 14 dpi.

To identify whether platelet presence in the retina was associated with damage to this structure, retinal thickness was measured in experimental and vehicle-only control mice. Coronal sections of the retina from normal, vehicle-only and MOG₃₅₋₅₅-induced groups at 14 dpi were analysed using ImageJ. A highly significant difference (p<0.001) in retinal thickness between MOG₃₅₋₅₅-induced and vehicle-only groups was found, with MOG₃₅₋₅₅-induced mice exhibiting increased thickness.

### Example 6: Inhibition of Platelet Function Eliminates EAE

To further confirm platelet involvement in the development of EAE, an alternative approach of interference with platelet biology in EAE induced mice was used. Briefly, a molecule targeting the ecto-nucleoside triphosphate diphosphohydrolase, also known as CD39, to activated platelets conjugated to a mutated non-functional scFv (scFVₘᵤₜ), a molecular comprising CD39 conjugated to a single-chain (ScFv) antibody against platelet marker CD41 (ScFv-CD39) were used (as described in Hohmann et al., 2013). Untreated mice were used as control.

ScFv-CD39 or ScFVmut-CD39 was administered by IV route at a dose of 1 μg/g body-weight to EAE induced mice. Antibody was administered on days 7, 9, 11, 13 and 15 post-induction as this was the regime that was successfully in eliminating EAE with platelet depletion (described above). Mice were weighed and monitored regularly for the development of clinical symptoms as previously described. Mean clinical score at end-point, mean cumulative clinical score, disease onset and duration between groups were used as parameters of comparisons between groups.

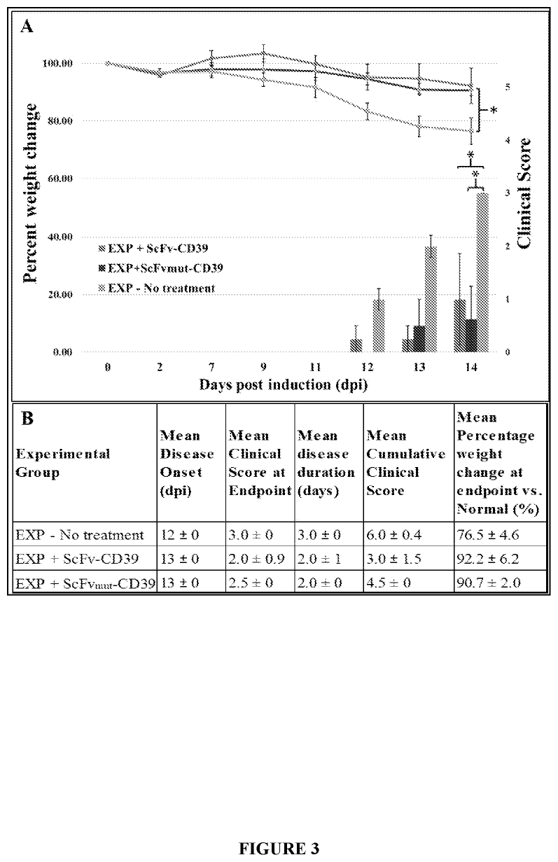

Significant amelioration of EAE was seen in the group that received the scFv-CD39 as well as the group that received the scFvₘᵤₜ-CD39 when compared to the untreated group (FIG. 3). Clinical score at end-point for both groups was significantly lower (p=0.023 and 0.016 respectively) and a significant difference in weight change was also observed (p=0.004 and 0.02). Interestingly, only one mouse from the group that received the scFVₘᵤₜ-CD39 developed clinical symptoms and only two mice from the group that received the scFv-CD39 developed clinical symptoms. The experiment had to be terminated at 14 DPI as the untreated control group had already reached the maximum allowed score of 3 by this time. These results confirm that platelets have an active function in the progression of EAE.

### Example 7: Platelet Depletion is Effective in Ameliorating Anxiety-Like Behaviour and Reducing the Pro-Inflammatory Environment in the Hippocampus in EAE

Female C57BL/6J mice (12-16 weeks old) were obtained from the Animal Resource Centre (Perth, Australia) and housed under standard conditions at 23° C. and 12:12 light:dark cycle, on standard rodent chow with food and water ad libitum.

MOG₃₃₋₅₅-induced EAE was performed as previously described. Briefly, on day 0 mice received two subcutaneous injections, each containing 100 μg MOG₃₅₋₅₅ peptide in 100 μL of PBS, in an equal volume of complete Freund's adjuvant (Sigma) supplemented with 4 mg/mL of Mycobacterium tuberculosis (Becton Dickinson). On days 0 and 2, mice received an intraperitoneal injection of 350 ng of pertussis toxin (PTx) (Sigma-Aldrich) in PBS. Clinical scores were given to monitor disease progression, as follows 0=no symptoms, limp tail=1, hind limb weakness=2, hind limb paralysis=3, ascending paralysis=4, and moribund=5. Control groups included vehicle-only (VO; omission of MOG₃₃₋₅₅) and normal mice.

There is a Direct Relationship Between Platelet Accumulation and that of Antigen-Specific T Cells in Neuroinflammation

Platelet numbers were estimated over the disease course in EAE-induced mice. Briefly, platelet counts were obtained from blood collected from the submandibular vein into K₂EDTA-coated blood Microtainers using a Sysmex XS-1000i (Sysmex America Inc. Mundelein, IL, USA) automated hematology analyzer. Platelet depletion (PD) with a polyclonal anti-GPIb alpha (CD42b) preparation (R300, Emfret Analytics, Eibelstadt, Germany) was achieved by IV administration, at seven days post induction (dpi) of EAE and at 0.5 pg/g body weight in 100 μL of PBS. Alternatively, as control, platelet depletion antibody was administered to vehicle-only mice. Platelet depletion was maintained by repeating the treatment every 48 h. An isotype antibody preparation (C301, Emfret Analytics) was administered to EAE-induced or vehicle-only groups as control, at the same times and dose.

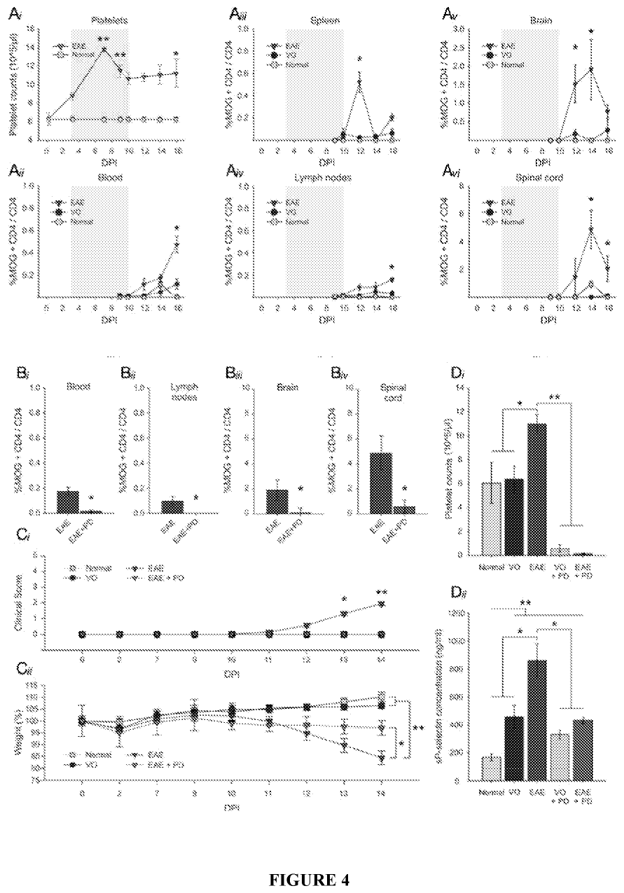

As shown in FIG. 4Ai, EAE-induced mice exhibited an increase in platelet numbers from 3 dpi, which reached a peak between 5 and 7 dpi, and were significantly elevated (p<0.01) above those of normal mice. Subsequently, a partial reduction in platelet numbers was observed, but these remained significantly above control levels (p<0.05) for the remainder of the disease course.

Concurrently, accumulation of MOG₃₅₋₅₅-specific T cells (expressed as the percentage of MOG₃₅₋₅₅-CD4+/total CD4⁺ cells) was estimated by intracellular cytokine staining (ICS) in blood, spleen, lymph nodes, brain, and spinal cord (FIG. 4Aii-Avi) in normal, vehicle-only, and EAE-induced mice. In normal and vehicle-only groups, no MOG₃₅₋₅₅-CD4⁺ cells were ever detected over the time course examined, in any tissue, as expected. In EAE-induced groups, in all of the tissues sampled, the earliest evidence of MOG₃₅₋₅₅-CD4⁺ cells were between 10 and 12 dpi, namely at least three to six days following the peak of platelet accumulation. Antigen-specific T cell accumulation displayed a monophasic pattern over the disease course in the spleen, brain, and spinal cord, with a peak at 12 dpi (spleen), or 14 dpi (brain and spinal cord), but continued to slowly accumulate in the blood and lymph nodes.

To determine whether a direct relationship exists between platelet and MOG₃₅₋₅₅-CD4⁺ accumulations, ICS was repeated in the presence of platelet depletion, which was induced from 7 dpi using an antibody against CD42b (or GP1b). This resulted in a reduction in platelet numbers in all control and experimental groups by above 96%, as well as maintenance of low platelet numbers by repeated anti-CD42b administration every 48 h. Evaluation of MOG₃₅₋₅₅-CD4⁺ accumulation at 14 dpi, showed significant reduction in blood, lymphoid organs and CNS tissues (FIG. 4Bi-Biv) in the EAE-induced/platelet depleted group, relative to EAE-induced/isotype antibody-treated group. This group did not develop disease symptoms, as demonstrated by absence of clinical scores by experimental end point, whilst their isotype antibody-treated counterparts reached a mean clinical score of 2.25±1.75 (FIG. 4Ci). Some weight loss was observed in EAE-induced/platelet depleted mice, but this was reduced relative to isotype antibody-treated mice (FIG. 4Cii). Confirmation of effective platelet depletion in these experiments was provided by the reduced platelet numbers in the EAE-induced/platelet depleted group relative to the isotype antibody-treated controls (FIG. 4Di), together with reduction in levels of sP-selectin, a major marker of platelet activation.

Determination of sP-selectin levels by ELISA (according to manufacture instructions) showed elevation in vehicle-only groups relative to normal mice, further augmented in EAE induced/isotype antibody-treated mice (FIG. 4Dii). These levels were restored to those of the vehicle-only group by platelet depletion.

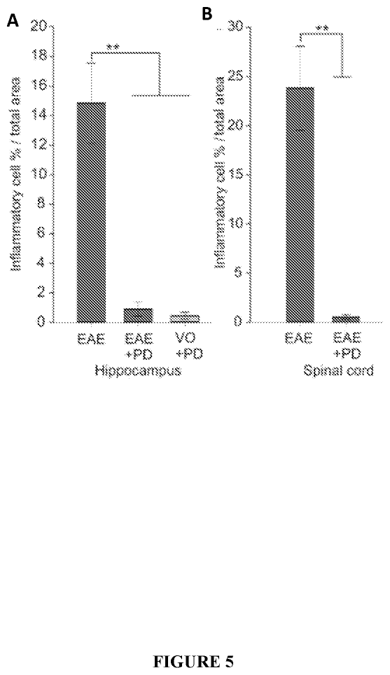

No inflammatory infiltration was detectable in EAE-induced/platelet depleted animals by H&E histological staining of brain (FIG. 5A) or spinal cord (FIG. 5B) by experimental end point, while severe inflammation was present throughout the whole of the neuraxis in isotype antibody-treated counterparts.

### Platelet Depletion Significantly Reduces Anxiety-Like Behaviour in EAE-Induced Mice

Depression and cognitive defects are regarded as primary disease manifestations, rather than secondary consequences of chronic illness. The elevated plus maze (EPM) is an accepted experimental paradigm to evaluate anxiety-like behaviour in rodents and is representative of depression.

To investigate the relationship between early platelet parenchymal entry and functional disturbance in the hippocampus (a CNS region associated with emotion and cognition) a single injection of platelet depleting antibody at 7 dpi, followed by the EPM test at 9 dpi was performed. Briefly, the EPM consists of a central platform (5×5 cm) with four branching arms (30×5 cm each) at right angles to each other, where one pair of opposite arms is walled and the other open. Following a single administration of platelet depleting antibody at 7 dpi, the test was conducted at 9 dpi in a soundproof room under dim red lighting (40-41 lux). Behaviour was recorded using a high definition webcam connected by a computer by an investigator blinded as to mouse identity and treatment conditions.

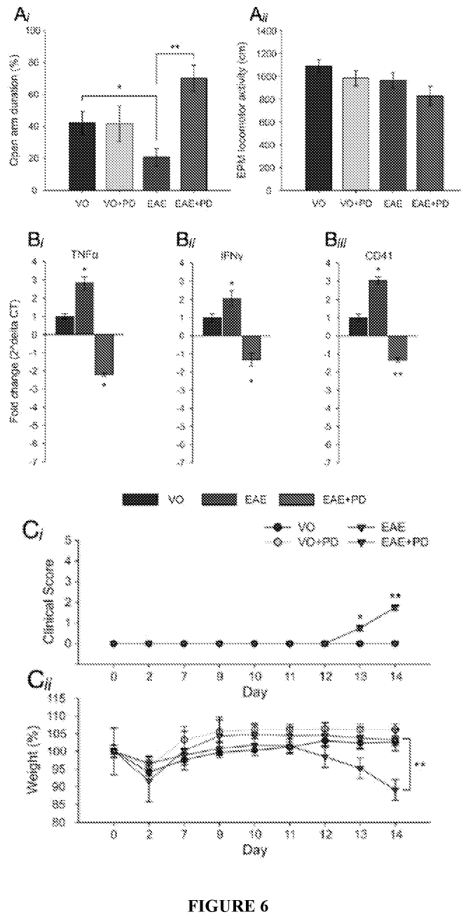

Results showed highly significantly reduced anxiety-like behaviour in the platelet depleted relative to the saline treated control groups, as evidenced by increased time spent in the open arms of the maze. There was no significant difference between vehicle-only/isotype antibody-treated and vehicle-only/platelet depleted groups in the percent time spent in the open arms of the maze (open arm duration (%), VO vs. VO+PD, 42.3±7.2 vs. 41.8±11.3, p=0.974) showing that platelet depletion in the absence of EAE induction is not associated with anxiety (FIG. 6Ai). On the other hand, a significant difference between the above groups and the EAE-induced/isotype antibody-treated group was demonstrated, showing that EAE induction is associated with anxiety-like behaviour (open arm duration (%), VO vs. EAE, 42.3±7.2 vs. 20.7±5.4, p=0.036) and that this effect is evident from the preclinical stage. However, there was a significant increase in the percent time spent in the open arms between the EAE-induced/isotype antibody-treated and EAE-induced/platelet depleted groups showing a beneficial effect of platelet depletion on anxiety-like behaviour (open arm duration (%), EAE vs. EAE+PD, 20.7±5.4 vs. 70.2±8.1, p=0.005). To identify potential early ambulatory difficulties, undetectable by visual observation, total distance covered is assessed (FIG. 6Aii). As shown in FIG. 6Aii, there was no significant difference in the total distance covered during the test period between groups showing absence of ambulatory difficulties at the time at which experimentation was conducted. At 9 dpi, all mice exhibited a clinical score of zero (FIG. 6Ci) and there were no significant differences in percent weight change between groups (FIG. 6Cii).

Following testing in the EPM, half of the mice in each control and experimental group were immediately humanely killed and the dorsal hippocampal region dissected for total RNA extraction, generation of cDNA and qPCR analysis of the pro-inflammatory cytokines TNF-α and IFN-γ and the platelet specific marker CD41 (FIG. 6Bi-Biii). In the case of TNF-α and IFN-γ, a significant difference was observed between the above groups and the EAE-induced/isotype antibody-treated group, showing that even by 9 dpi, a severe inflammatory environment was present in the hippocampal region. This pro-inflammatory environment was associated with the presence of platelets as shown by the significant difference in CD41 expression levels between the same groups. Platelet depletion resulted in the significant reduction in expression of the pro-inflammatory and platelet markers. As an additional control, the remaining mice in each group were maintained on the treatment assigned to their group and were humanely killed at 14 dpi. Only the EAE-induced/isotype antibody group developed EAE (clinical score of 2.0±0.1 at 14 dpi, (FIG. 6Ci), whilst vehicle-only/isotype antibody, vehicle-only/platelet depleted and EAE-induced/platelet depleted groups remained clinical score free, nor did they exhibit weight loss (weight (%) from 0 dpi (FIG. 6Cii).

Anxiety-Like Behaviour and the Pro-Inflammatory Environment in the Hippocampus are Characterized by Platelet-Neuron Association

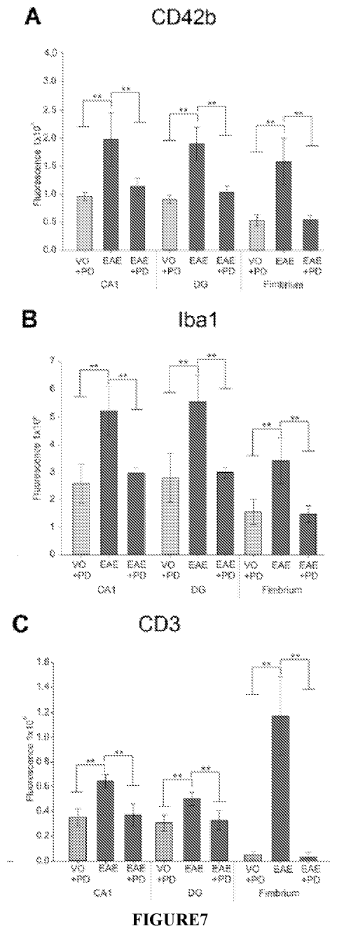

To further investigate the relationship between anxiety-like behavior, parenchymal platelet accumulation and lymphocytic infiltration, immunochemistry was performed with tissues from mice used in the EPM test (data not shown). At 9 and 14 dpi, combined anti-CD42b and anti-MAP2 revealed extensive platelet accumulation in the EAE-induced/isotype antibody-treated group only, where they were particularly prominent in the CA1 region, dentate gyrus and fimbrium (data not shown). In the fimbrium, diffuse platelet distribution was observed; on the other hand, in the CA1 region and dentate gyrus platelets appeared to associate principally with neuronal cell bodies. Quantification of immunofluorescence signals confirmed the significant difference in platelet accumulation between EAE-induced/isotype antibody-treated and EAE-induced/platelet depleted groups and absence of significance between EAE-induced/platelet depleted and vehicle-only/platelet depleted groups (FIG. 7). Combined anti-Ibal and anti-CD3 were used to identify parameters of inflammation relative to platelet accumulation. Ibal reactivity was identified by larger cell bodies and more complex branching of processes throughout the whole of the dorsal hippocampus, in the EAE-induced/isotype antibody-treated group only, from 9 dpi. CD3 was identified only at 14 dpi and the presence of CD3 positive cells was restricted to the fimbrium and adjacent choroid plexus. Quantification of immunofluorescence signals confirmed the significant difference in Ibal and CD3 levels between EAE-induced/isotype antibody-treated and EAE-induced/platelet depleted groups and absence of significance between EAE-induced/platelet depleted and vehicle-only/platelet depleted groups (FIG. 7). Taken together, these data showed that the strong platelet presence in the hippocampal formation in the EAE/isotype antibody-treated group from the pre-clinical stage was not associated with inflammatory cell infiltration.

### Example 8: High-Dose scFv-CD39 Treatment is Efficacious

Given the demonstrated efficacy of scFv-CD39 at 0.4 mg/kg, mice were administered high dose treatment of scFV-CD39 at 1.2 μg/g as described above at 48 hour intervals. Control animals were treated with scFvₘᵤₜ-CD39 using the same parameters. Animals were assessed for clinical symptoms and weighed daily until day 20, which represents humane end point for animals which do develop the disease. At experimental end point tissues were collected for quantification of disease activity and potentially haemorrhage, by histological and immunochemical techniques.

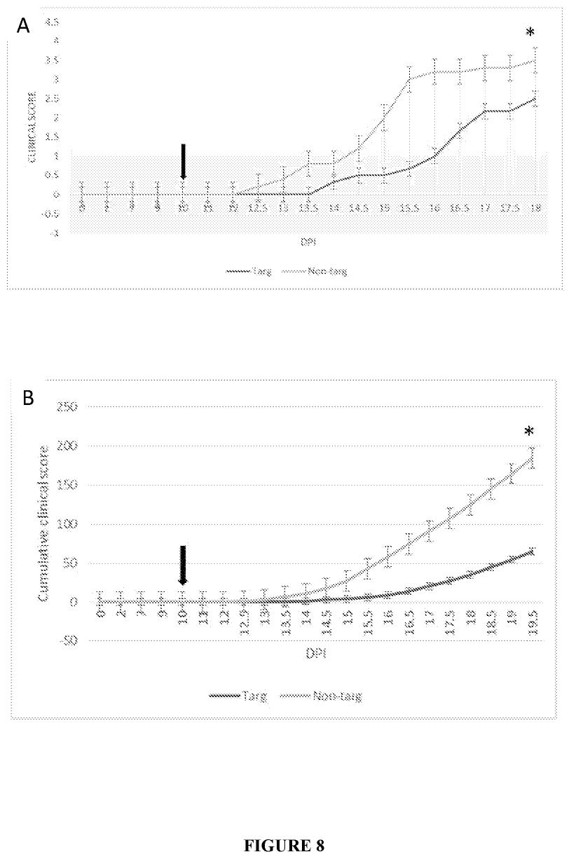

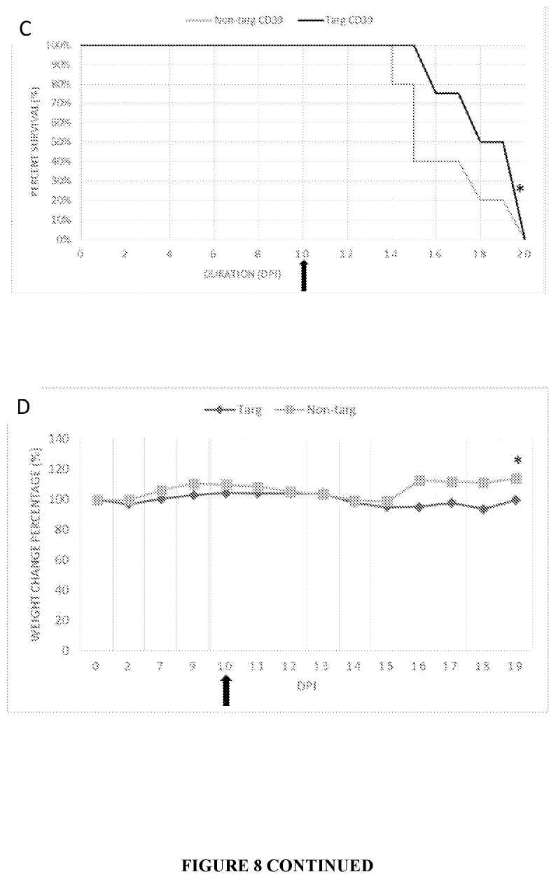

As shown in FIG. 8, significant differences between the scFv-CD39 and scFvₘᵤₜ-CD39 groups in disease severity and progression in all clinical measures, including clinical score over time (A), cumulative scores over time (B), survival curve (C) and weight loss over time (D) were observed. No adverse events were recorded. These data confirm the safety and efficacy in EAE, when treatment is initiated upon the earliest evidence of disease at 1.2 μg/g body weight.

### Example 9: scFv-CD39 Treatment after Disease Onset is Efficacious

This Example compared prophylactic and therapeutic drug efficacy of scFv-CD39 in the EAE mouse model described in Example 1, by staggering the timing of treatment initiation from day 7 to day 12 post disease initiation. Disease onset is around day 10-12 and is first manifested by a sudden and rapidly escalating weight loss of 10-12%. The same drug dosage of 0.4 mg/kg every 48 hours was administered.

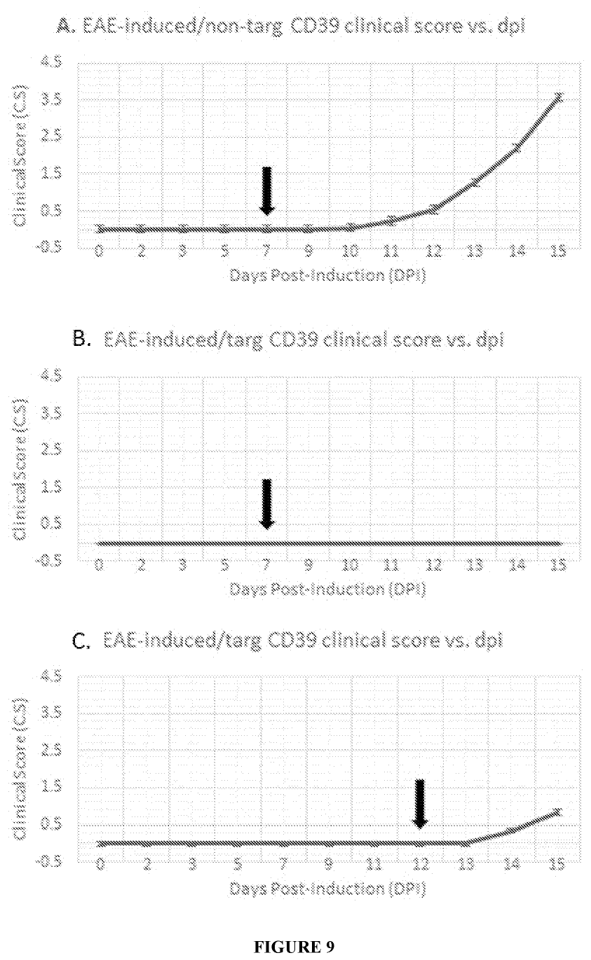

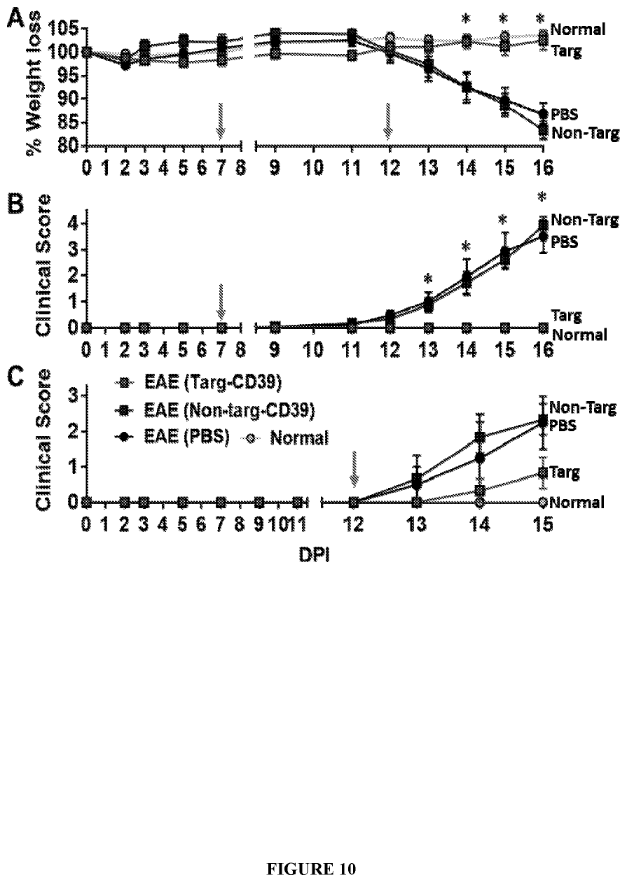

As per the previous Examples, prophylactic treatment (treatment started on day 7 post disease initiation) using scFv-CD39 completely prevented disease development (FIG. 9B and FIG. 10B), whereas the scFvₘᵤₜ-CD39 treated mice displayed rapid progression (FIG. 9A). scFv-CD39 treatment after disease onset (treatment started at day 12 post disease initiation) resulted in delayed disease progression and resulted in a significantly milder disease profile.

No significant weight loss was detected with scFv-CD39 therapeutic treatment (treatment started on day 12 post disease initiation), whereas scFvₘᵤₜ-CD39 treated mice and saline treated mice showed severe weight loss from days 11 to 12 (FIG. 10A).

FIG. 10C confirms that scFv-CD39 therapeutic treatment (treatment started on day 12 post disease initiation) delayed disease onset and slowed the progression of disease compared to scFvₘᵤₜ-CD39 treated mice and saline treated mice.

### Example 10: A Milder EAE Disease Model Demonstrates Hallmarks of MS

A new EAE disease model, exhibiting milder disease progression, was generated to be more comparative to MS. The mild disease model was induced using a lower amount of pertussis toxin (300 ng vs 350 ng in the model described in Example 1). Also, incomplete Freund's adjuvant (IFA) was used rather than the complete Freund's adjuvant (CFA) used in the model described in Example 1, thereby reducing the amount of heat-inactivated Mycobacterium tuberculosis administered to a maximum of 4 mg/mL in the mild model relative to a minimum of 6 mg/mL in the standard model.

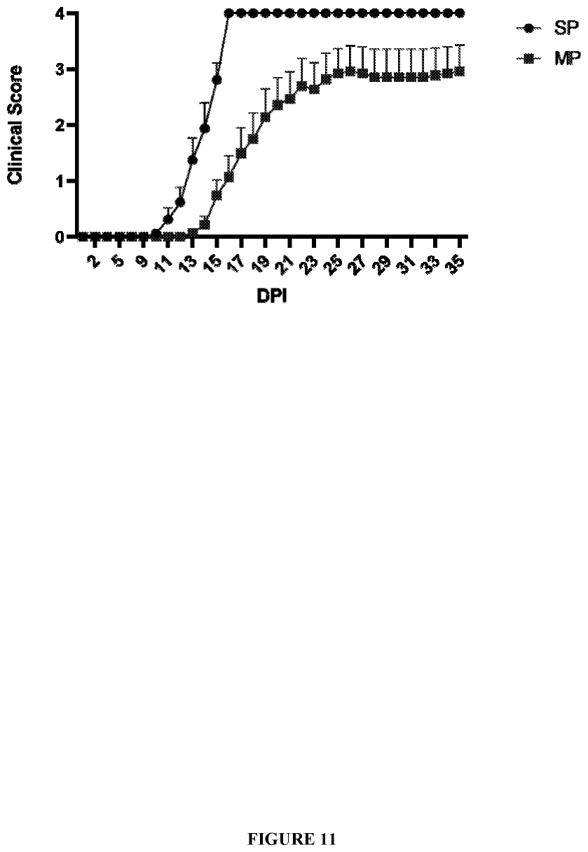

As shown in FIG. 11, the mild protocol results in a delayed disease onset (Clinical onset dpi: 14±0.15 compared to 11±0.21 for standard protocol) and lower clinical score at the peak of disease (peak clinical score at 35 dpi compared to 15 for standard protocol). Therefore the mice can be kept in experimentation for a longer period. In this regard, the humane end point is reached when the clinical score exceeds 3. Therefore, the experimental window was increased from 4 days (days 12-16) to over 19 days (days 16 to >day 35).

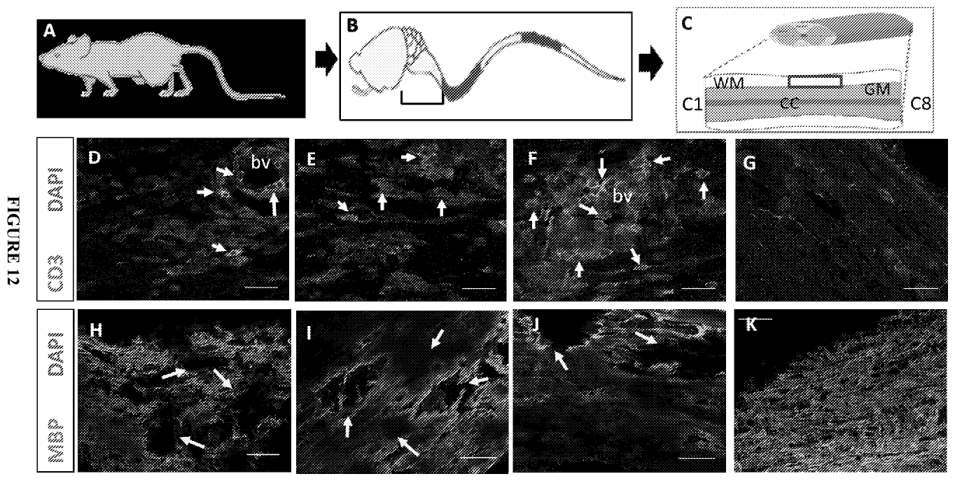

Furthermore, FIG. 12 shows that the disease pathology in the new mild model exhibits the same hallmarks as the standard model and consequently, recapitulates critical facets of MS. Specifically, FIG. 12 demonstrates that the mild disease model displays presence of autoimmune cells in the spinal cord (FIG. 12D-F) and destruction of myelin (FIG. 12H-J), which are the hallmarks of MS.

### Example 11: scFv-CD39 Treatment after Disease Onset is Efficacious in the Mild Disease Model

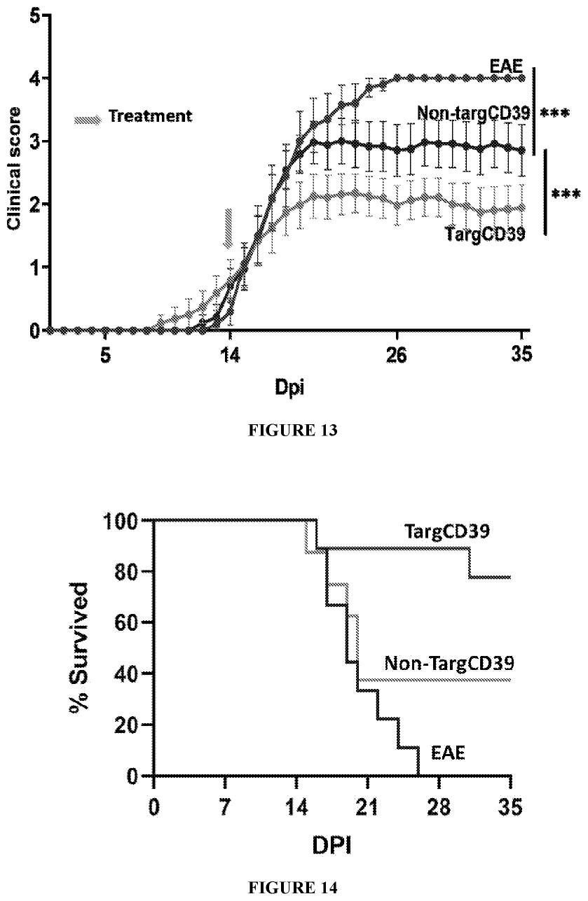

FIG. 13 shows that treatment of mice in which mild EAE was induced resulted in highly significant reduction in mean clinical scores with scFv-CD39 therapeutic treatment, compared to scFvₘᵤₜ-CD39 treated mice and untreated mice. In this Example, scFv-CD39 treatment was initiated when mice reached a clinical score between 1 to 2 and had a minimum of 10-12% weight loss. The drug dosage of 0.4 mg/kg every 48 hours was used.

This Example demonstrates the beneficial effects of scFv-CD39 therapeutic treatment on disease progression and highly significant differences in mean clinical scores, at experimental end point, between the three experimental groups.

Furthermore, the survival curves in FIG. 14 show that 80% of mice survive past day 35 with scFv-CD39 treatment, 40% with scFvₘᵤₜ-CD39 and 0% without drug treatment. Out of the 80% scFv-CD39-treated surviving animals, 78% exhibited inhibition of progression, associated with moderate to marked return to minimal clinical scores.

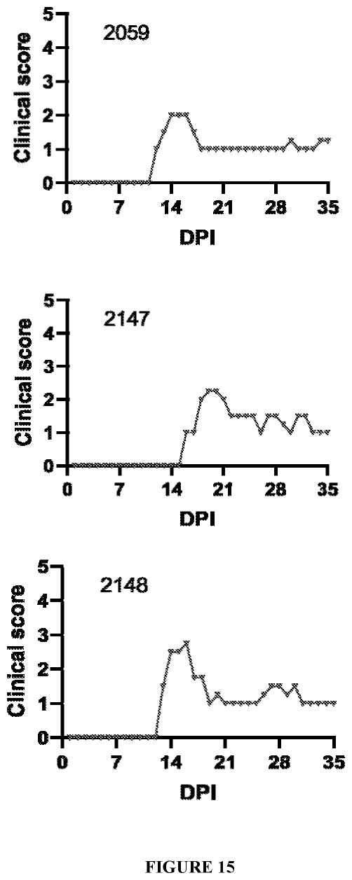

Selected individual mouse clinical score profiles are shown in FIG. 15. The majority of animals (80-90%) had a profile similar to mice #2059, 2147, 2148 (FIG. 15) and showed inhibition of disease progress, followed by remarkable recovery to clinical score 1 (limp tail only, fully ambulatory animals). Recovery was also observed even in mice (such as mouse #2148, FIG. 15) which had reached a maximum clinical score of 3, i.e., very severe disease with hind limb paralysis.

### Example 12: Production of scFv-CD39 Fusion Proteins

Production of scFv-CD39 fusions in mammalian cells was performed using human kidney cells (HEK293F) in suspension culture after transfection with polyethylenimine (Polyscience Inc., Germany). DNA plasmid encoding scFv-CD39 was diluted to a ratio of 1:4 with polyethylenimine (PEI) for transfection. 24 hours prior to transfection, HEK293F cells were diluted with Freestyle 293 expression medium (Invitrogen) to a concentration of 1×10⁶ cells/ml. The cell density was approximately 2×10⁶ cells/ml at time of transfection and the viability greater than 95%. The amount of Freestyle 293 expression medium to the PBS mixture of DNA and PEI was at a ratio of 9:1.

Appropriate amount of cell culture medium was transferred into a shaker flask and placed in an incubator at 37° C., shaking at 110 rpm. 1 pg/ml of DNA plasmid was added to pre-warmed (37° C.) PBS and vortexed gently. PEI was added to the concentration of 3 pg/ml, and vortexed shortly. The mixture was incubated for 15 min at RT. The DNA-PEI mixture was added to the pre-warmed medium while swirling gently. Glucose was added to a final concentration of 6 g/L. The flask was returned to the incubator and cultured at 37° C., with 5% CO2, shaking at 110-140 rpm. The culture was supplemented with 5 g/L Lupin and 0.2 mM butyric acid after one day. At days 3, 5 and 7 after transfection, the culture was supplemented with 2 mM L-glutamine. At day 5, the culture was again supplemented with 5 g/L Lupin. The glucose level was maintained at a final concentration of 5-6 g/L. The cells were harvested when viability was 40-50%. The cells were centrifuged at 3000 g for 15 min at 4° C. and supernatant was collected for protein purification. Proteins were purified with a nickel-based metal affinity chromatography column, Ni-NTA column (Invitrogen), according to the manufacturer's instructions. Fractions of 1 ml were collected and dialyzed against PBS.

The following scFv-CD39 fusion proteins are produced using these methods:

SEQ ID NO: 6
MAEVQLVESGGGLVQPGGSLRLSCAASGFMFSRYAMSWVRQAPGKGPEWV
SGISGSGGSTYYADSVKGRFTVSRDNSKNTLYLQMNSLRAEDTAVYYCAR
IFTHRSRGDVPDQTSFDYWGQGTLVTVSSGSASAPKLEEGEFSEARVSSE
LTQDPAVSVALGQTVRITCQGDSLRNFYASWYQQKPGQAPTLVIYGLSKR
PSGIPDRFSASSSGNTASLTITGAQAEDEADYYCLLYYGGGQQGVFGGGT
KLTVLRQPKAAPSVTLFPPSSAAATQNKALPENVKYGIVLDAGSSHTSLY
IYKWPAEKENDTGVVHQVEECRVKGPGISKFVQKVNEIGIYLTDCMERAR
EVIPRSQHQETPVYLGATAGMRLLRMESEELADRVLDVVERSLSNYPFDF
QGARIITGQEEGAYGWITINYLLGKFSQKTRWFSIVPYETNNQETFGALD
LGGASTQVTFVPQNQTIESPDNALQFRLYGKDYNVYTHSFLCYGKDQALW
QKLAKDIQVASNEILRDPCFHPGYKKVVNVSDLYKTPCTKRFEMTLPFQQ
FEIQGIGNYQQCHQSILELFNTSYCPYSQCAFNGIFLPPLQGDFGAFSAF
YFVMKFLNLTSEKVSQEKVTEMMKKFCAQPWEEIKTSYAGVKEKYLSEYC
FSGTYILSLLLQGYHFTADSWEHIHFIGKIQGSDAGWTLGYMLNLTNMIP
AEQPLSTPLSHST

where:
Uppercase regular text corresponds to the anti-GPIIb/IIIa (CD41) scFv;
GSASAPKLEEGEFSEARVS [SEQ ID NO:29] is a flexible linker in the scFv;
AAA is a flexible linker; and
Uppercase underlined text corresponds to the amino acid sequence of the extracellular domain of CD39.

SEQ ID NO: 24
EVQLVESGGGLVQPGGSLRLSCAASGFMFSRYAMSWVRQAPGKGPEWVSG
ISGSGGSTYYADSVKGRFTVSRDNSKNTLYLQMNSLRAEDTAVYYCARIF
THRSRGDVPDQTSFDYWGQGTLVTVSSGSASAPKLEEGEFSEARVSSELT
QDPAVSVALGQTVRITCQGDSLRNFYASWYQQKPGQAPTLVIYGLSKRPS
GIPDRFSASSSGNTASLTITGAQAEDEADYYCLLYYGGGQQGVFGGGTKL
TVLRQPKAAPSVTLFPPSSAAATQNKALPENVKYGIVLDAGSSHTSLYIY
KWPAEKENDTGVVHQVEECRVKGPGISKFVQKVNEIGIYLTDCMERAREV
IPRSQHQETPVYLGATAGMRLLRMESEELADRVLDVVERSLSNYPFDFQG
ARIITGQEEGAYGWITINYLLGKFSQKTRWFSIVPYETNNQETFGALDLG
GASTQVTFVPQNQTIESPDNALQFRLYGKDYNVYTHSFLCYGKDQALWQK
LAKDIQVASNEILRDPCFHPGYKKVVNVSDLYKTPCTKRFEMTLPFQQFE
IQGIGNYQQCHQSILELFNTSYCPYSQCAFNGIFLPPLQGDFGAFSAFYF
VMKFLNLTSEKVSQEKVTEMMKKFCAQPWEEIKTSYAGVKEKYLSEYCFS
GTYILSLLLQGYHFTADSWEHIHFIGKIQGSDAGWTLGYMLNLTNMIPAE
QPLSTPLSHST

where:
Uppercase regular text corresponds to the anti-GPIIb/IIIa (CD41) scFv;
GSASAPKLEEGEFSEARVS [SEQ ID NO:29] is a flexible linker in the scFv;
AAA is a flexible linker; and
Uppercase underlined text corresponds to the amino acid sequence of the extracellular domain of CD39.

SEQ ID NO: 25
EVQLVESGGGLVQPGGSLRLSCAASGFMFSRYAMSWVRQAPGKGPEWVSG
ISGSGGSTYYADSVKGRFTVSRDNSKNTLYLQMNSLRAEDTAVYYCARIF
THRSRGDVPDQTSFDYWGQGTLVTVSSGSASAPKLEEGEFSEARVSSELT
QDPAVSVALGQTVRITCQGDSLRNFYASWYQQKPGQAPTLVIYGLSKRPS
GIPDRFSASSSGNTASLTITGAQAEDEADYYCLLYYGGGQQGVFGGGTKL
TVLAAATQNKALPENVKYGIVLDAGSSHTSLYIYKWPAEKENDTGVVHQV
EECRVKGPGISKFVQKVNEIGIYLTDCMERAREVIPRSQHQETPVYLGAT
AGMRLLRMESEELADRVLDVVERSLSNYPFDFQGARIITGQEEGAYGWIT
INYLLGKFSQKTRWFSIVPYETNNQETFGALDLGGASTQVTFVPQNQTIE
SPDNALQFRLYGKDYNVYTHSFLCYGKDQALWQKLAKDIQVASNEILRDP
CFHPGYKKVVNVSDLYKTPCTKRFEMTLPFQQFEIQGIGNYQQCHQSILE
LFNTSYCPYSQCAFNGIFLPPLQGDFGAFSAFYFVMKFLNLTSEKVSQEK
VTEMMKKFCAQPWEEIKTSYAGVKEKYLSEYCFSGTYILSLLLQGYHFTA
DSWEHIHFIGKIQGSDAGWTLGYMLNLTNMIPAEQPLSTPLSHST

where:
Uppercase regular text corresponds to the anti-GPIIb/IIIa (CD41) scFv;
GSASAPKLEEGEFSEARVS [SEQ ID NO:29] is a flexible linker in the scFv;
AAA is a flexible linker; and
Uppercase underlined text corresponds to the amino acid sequence of the extracellular domain of CD39.

SEQ ID NO: 26
MAEVQLVESGGGLVQPGGSLRLSCAASGFMFSRYAMSWVRQAPGKGPEWV
SGISGSGGSTYYADSVKGRFTVSRDNSKNTLYLQMNSLRAEDTAVYYCAR
IFTHRSRGDVPDQTSFDYWGQGTLVTVSSGSASAPKLEEGEFSEARVSSE
LTQDPAVSVALGQTVRITCQGDSLRNFYASWYQQKPGQAPTLVIYGLSKR
PSGIPDRFSASSSGNTASLTITGAQAEDEADYYCLLYYGGGQQGVFGGGT
KLTVLAAATQNKALPENVKYGIVLDAGSSHTSLYIYKWPAEKENDTGVVH
QVEECRVKGPGISKFVQKVNEIGIYLTDCMERAREVIPRSQHQETPVYLG
ATAGMRLLRMESEELADRVLDVVERSLSNYPFDFQGARIITGQEEGAYGW
ITINYLLGKFSQKTRWFSIVPYETNNQETFGALDLGGASTQVTFVPQNQT
IESPDNALQFRLYGKDYNVYTHSFLCYGKDQALWQKLAKDIQVASNEILR
DPCFHPGYKKVVNVSDLYKTPCTKRFEMTLPFQQFEIQGIGNYQQCHQSI
LELFNTSYCPYSQCAFNGIFLPPLQGDFGAFSAFYFVMKFLNLTSEKVSQ
EKVTEMMKKFCAQPWEEIKTSYAGVKEKYLSEYCFSGTYILSLLLQGYHF
TADSWEHIHFIGKIQGSDAGWTLGYMLNLTNMIPAEQPLSTPLSHST

where:
Uppercase regular text corresponds to the anti-GPIIb/IIIa (CD41) scFv;
GSASAPKLEEGEFSEARVS [SEQ ID NO:29] is a flexible linker in the scFv;
AAA is a flexible linker; and
Uppercase underlined text corresponds to the amino acid sequence of the extracellular domain of CD39.

SEQ ID NO: 27
METDTLLLWVLLLWVPGSTGDAAQPARRAVRSLVPSSDPLQCGGILHHHH
HHHHRRAMAEVQLVESGGGLVQPGGSLRLSCAASGFMFSRYAMSWVRQAP
GKGPEWVSGISGSGGSTYYADSVKGRFTVSRDNSKNTLYLQMNSLRAEDT
AVYYCARIFTHRSRGDVPDQTSFDYWGQGTLVTVSSGSASAPKLEEGEFS
EARVSSELTQDPAVSVALGQTVRITCQGDSLRNFYASWYQQKPGQAPTLV
IYGLSKRPSGIPDRFSASSSGNTASLTITGAQAEDEADYYCLLYYGGGQQ
GVFGGGTKLTVLRQPKAAPSVTLFPPSSAAATQNKALPENVKYGIVLDAG
SSHTSLYIYKWPAEKENDTGVVHQVEECRVKGPGISKFVQKVNEIGIYLT
DCMERAREVIPRSQHQETPVYLGATAGMRLLRMESEELADRVLDVVERSL
SNYPFDFQGARIITGQEEGAYGWITINYLLGKFSQKTRWFSIVPYETNNQ
ETFGALDLGGASTQVTFVPQNQTIESPDNALQFRLYGKDYNVYTHSFLCY
GKDQALWQKLAKDIQVASNEILRDPCFHPGYKKVVNVSDLYKTPCTKRFE
MTLPFQQFEIQGIGNYQQCHQSILELFNTSYCPYSQCAFNGIFLPPLQGD
FGAFSAFYFVMKFLNLTSEKVSQEKVTEMMKKFCAQPWEEIKTSYAGVKE
KYLSEYCFSGTYILSLLLQGYHFTADSWEHIHFIGKIQGSDAGWTLGYML
NLTNMIPAEQPLSTPLSHSTTRGGPEQKLISEEDLNSAVDHHHHHH

where:
Uppercase italic text corresponds to a leader sequence [SEQ ID NO:30] used for expression of the construct;
Uppercase regular text corresponds to the anti-GPIIb/IIIa (CD41) scFv;
GSASAPKLEEGEFSEARVS [SEQ ID NO:29] is a flexible linker in the scFv;
AAA is a flexible linker;
Uppercase underlined text corresponds to the amino acid sequence of the extracellular domain of CD39; and
His tags and a C-myc tag are underlined and italicized.

SEQ ID NO: 28
METDTLLLWVLLLWVPGSTGDAAQPARRAVRSLVPSSDPLQCGGILHHHH
HHHHRRAMAEVQLVESGGGLVQPGGSLRLSCAASGFMFSRYAMSWVRQAP
GKGPEWVSGISGSGGSTYYADSVKGRFTVSRDNSKNTLYLQMNSLRAEDT
AVYYCARIFTHRSRGDVPDQTSFDYWGQGTLVTVSSGSASAPKLEEGEFS
EARVSSELTQDPAVSVALGQTVRITCQGDSLRNFYASWYQQKPGQAPTLV
IYGLSKRPSGIPDRFSASSSGNTASLTITGAQAEDEADYYCLLYYGGGQQ
GVFGGGTKLTVLAAATQNKALPENVKYGIVLDAGSSHTSLYIYKWPAEKE
NDTGVVHQVEECRVKGPGISKFVQKVNEIGIYLTDCMERAREVIPRSQHQ
ETPVYLGATAGMRLLRMESEELADRVLDVVERSLSNYPFDFQGARIITGQ
EEGAYGWITINYLLGKFSQKTRWFSIVPYETNNQETFGALDLGGASTQVT
FVPQNQTIESPDNALQFRLYGKDYNVYTHSFLCYGKDQALWQKLAKDIQV
ASNEILRDPCFHPGYKKVVNVSDLYKTPCTKRFEMTLPFQQFEIQGIGNY
QQCHQSILELFNTSYCPYSQCAFNGIFLPPLQGDFGAFSAFYFVMKFLNL
TSEKVSQEKVTEMMKKFCAQPWEEIKTSYAGVKEKYLSEYCFSGTYILSL
LLQGYHFTADSWEHIHFIGKIQGSDAGWTLGYMLNLTNMIPAEQPLSTPL
SHSTTRGGPEQKLISEEDLNSAVDHHHHHH

where:
Uppercase italic text corresponds to a leader sequence [SEQ ID NO:30] used for expression of the construct;
Uppercase regular text corresponds to the anti-GPIIb/IIIa (CD41) scFv;
GSASAPKLEEGEFSEARVS [SEQ ID NO:29] is a flexible linker in the scFv;
AAA is a flexible linker;
Uppercase underlined text corresponds to the amino acid sequence of the extracellular domain of CD39; and
His tags and a C-myc tag are underlined and italicized.

All of the above fusion proteins comprised the same CDR sequences in the scFv region, namely:

V_{H} CDR1:
[SEQ ID NO: 15]
RYAMS
V_{H} CDR2:
[SEQ ID NO: 16]
GISGSGGSTYYADSVKG
V_{H} CDR3:
[SEQ ID NO: 17]
CARIFTHRSRGDVPDQTSFDY
V_{L} CDR1:
[SEQ ID NO: 18]
QGDSLRNFYAS
V_{L} CDR2:
[SEQ ID NO: 19]
GLSKRPS
V_{L} CDR3:
[SEQ ID NO: 20]
LLYYGGGQQGV

### Example 13: Bioluminometric Determination of CD39 Function

A malachite green phosphate assay kit from Gentaur was used to determine the enzymatic activity of scFv-CD39, scFvₘᵤₜ-CD39, and commercially available recombinant human CD39 (R&D Systems) by measuring the release of phosphate during the conversion of ADP to adenosine 5′-monophosphate (AMP). For every molecule of ADP that is converted into AMP, 1 molecule of phosphate is released. Proteins were incubated at 37° C. with a series of ADP concentrations from 0 to 100 mM. The reactions were stopped at several time points from 0 to 120 minutes. The samples were measured at a wavelength of 650 nm on a Victor 3V Multi-label counter (PerkinElmer). A standard series of phosphate concentrations was used to convert raw data to the amount of AMP generated for each of the proteins. The amount of AMP generated vs time of incubation was then used to obtain the velocity of the reaction for each substrate (ADP) concentration. These velocity values were then graphed against the substrate (ADP) starting concentration to obtain Vmax and Km.

In PBS samples, serial concentrations of scFv-CD39, scFvₘᵤₜ-CD39 and commercially available recombinant human CD39 from Abcam (UK) were incubated with 100 μM ADP for 10 min. Remaining ADP was then converted to ATP by the pyruvate kinase reaction: To 100 μl EDTA-PBS, 33 μl solution containing 40 U/ml pyruvate kinase, 4 μM phosphoenolpyruvate (PEP; Sigma-Aldrich, Australia), 10 mM KCl, and 40 mM MgSO4 in 40 mM tricine buffer (pH 7.75) was added. After 5 min incubation, each sample was divided into two aliquots of 50 μl each. ATP (repre-senting the non-hydrolized remaining ADP) was determined in a bioluminescence assay using a microplate luminometer (Berthold MicroLumatPlus, Australia) by adding 50 μl luciferase reagent (ATP bioluminescence assay kit CLS II; Roche, Germany) to each sample. Hydrolized ADP levels were determined by subtracting the obtained ADP values from the 100 μM ADP starting concentration. Standard samples containing different concentrations of ADP in EDTA-PBS were also measured in order to establish a standard curve for the back-calculation of ADP levels.

The results are shown in Table 3 below.

TABLE 3
CD39 activity assay results

| Parameter | Soluble CD39 | scFv-CD39 | scFVₘᵤₜ-CD39 |
|---|---|---|---|
| Vmax (pmoles/min) | 18.36 ± 2.100 | 11.66 ± 2.242 | 9.834 ± 1.171 |
| Km (μM) | 40.60 ± 10.70 | 49.86 ± 20.35 | 36.60 ± 10.47 |
| Amount of protein | 3.333 | 6.667 | 3.333 |
| employed in assay | | | |
| (ng) | | | |
| Amount of actual | 3.333 | 5.352 | 2.059 |
| CD39 enzyme | | | |
| component in assay | | | |
| Specific activity | 5508 | 2833 | 4778 |
| (pmoles/min/μg of the | | | |
| CD39 component) | | | |
| adjusted for the CD39 | | | |
| component | | | |

### Example 14: In Vitro Activated Platelet Targeting of scFv-CD39

Binding of scFv-CD39 to activated human platelets was evaluated by flow cytometry: 0.1 mg/mL of scFv-CD39, 0.2 mg/mL of scFvₘᵤₜ-CD39 (both activity matched), and 0.038 mg/mL of anti-CD41 scFv (equimolar amount of the scFv in scFv-CD39) were tested.

Citrated blood from volunteers was centrifuged at 180 g for 10 minutes. The platelet-rich plasma (PRP) was collected and stored at 37° C. The remainder (infranatant) was centrifuged at 2500 g for 10 minutes, and its supernatant was collected as platelet-poor plasma (PPP).

PRP was diluted 1:20 in phosphate-buffered saline (PBS; 100 mg/L calcium chloride, 100 mg/L magnesium chloride). To investigate the binding of scFv-CD39 constructs, the diluted PRP (45 mL) was either preincubated with a final concentration of 10 mM ADP or 5 mL of PBS for 15 minutes before addition of the constructs. The binding was then determined via a Penta-His Alexa Fluor-488-conjugated monoclonal antibody (Qiagen). To investigate the ecto-nucleoside triphosphate diphosphohydrolase efficiency, PRP was preincubated with scFv-CD39 constructs before administration of 20 mM ADP. Platelet activation status was measured by a phycoerythrin (PE)-labeled anti-P-selectin antibody (BD Bioscience). Samples were fixed using 13 Cellfix (BD Bioscience) and analyzed on a FACS Calibur (BD Bioscience).

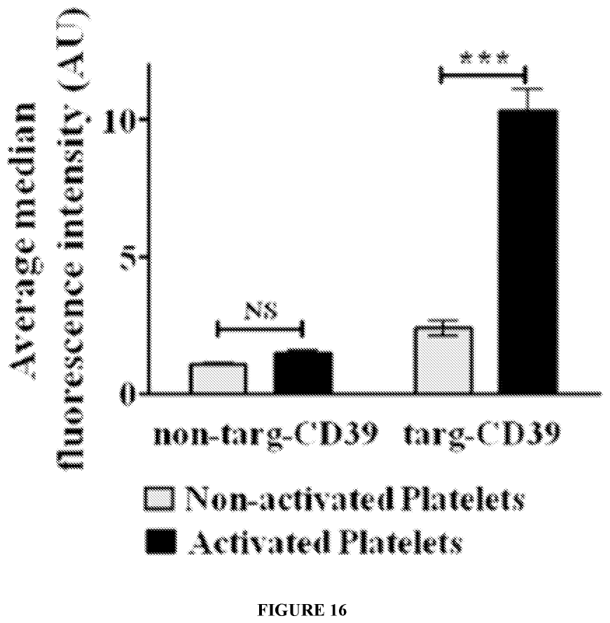

FIG. 16 shows that scFv-CD39 binds selectively to activated platelets but not to non-activated platelets. Flow cytometry also revealed significantly more binding of scFv-CD39 to activated platelets compared with the scFvₘᵤₜ-CD39 control (FIG. 16), which demonstrated only background binding to activated platelets. Neither scFv-CD39 nor scFvₘᵤₜ-CD39 showed significant binding to resting non-activated platelets (FIG. 16). scFvₘᵤₜ-CD39 also does not show binding to activated platelets (FIG. 16).

### Example 15: A scFv-CD39 Fusion Comprising Human Serum Albumin

A scFv-CD39 fusion comprising human serum albumin (scFv-HSA-CD39) was constructed using PCR and restriction enzyme cloning. The HSA sequence (SEQ ID NO: 31) was inserted between the scFv and the CD39 sequences. The sequence of the resulting scFv-HSA-CD39 construct is shown below:

SEQ ID NO: 32
METDTLLLWVLLLWVPGSTGDAAQPARRAMAEVQLVESGGGLVQPGGSL
RLSCAASGFMFSRYAMSWVRQAPGKGPEWVSGISGSGGSTYYADSVKGR
FTVSRDNSKNTLYLQMNSLRAEDTAVYYCARIFTHRSRGDVPDQTSFDY
WGQGTLVTVSSGSASAPKLEEGEFSEARVSSELTQDPAVSVALGQTVRI
TCQGDSLRNFYASWYQQKPGQAPTLVIYGLSKRPSGIPDRFSASSSGNT
ASLTITGAQAEDEADYYCLLYYGGGQQGVFGGGTKLTVLRQPKAAPSVT
LFPPSSAISMDAHKSEVAHRFKDLGEENFKALVLIAFAQYLQQCPFEDH
VKLVNEVTEFAKTCVADESAENCDKSLHTLFGDKLCTVATLRETYGEMA
DCCAKQEPERNECFLQHKDDNPNLPRLVRPEVDVMCTAFHDNEETFLKK
YLYEIARRHPYFYAPELLFFAKRYKAAFTECCQAADKAACLLPKLDELR
DEGKASSAKQRLKCASLQKFGERAFKAWAVARLSQRFPKAEFAEVSKLV
TDLTKVHTECCHGDLLECADDRADLAKYICENQDSISSKLKECCEKPLL
EKSHCIAEVENDEMPADLPSLAADFVESKDVCKNYAEAKDVFLGMFLYE
YARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLG
KVGSKCCKHPEAKRMPCAEDYLSVVLNQLCVLHEKTPVSDRVTKCCTES
LVNRRPCFSALEVDETYVPKEFNAETFTFHADICTLSEKERQIKKQTAL
VELVKHKPKATKEQLKAVMDDFAAFVEKCCKADDKETCFAEEGKKLVAA
SQAALGLGGGGGGAAATQNKALPENVKYGIVLDAGSSHTSLYIYKWPAE
KENDTGVVHQVEECRVKGPGISKFVQKVNEIGIYLTDCMERAREVIPRS
QHQETPVYLGATAGMRLLRMESEELADRVLDVVERSLSNYPFDFQGARI
ITGQEEGAYGWITINYLLGKFSQKTRWFSIVPYETNNQETFGALDLGGA
STQVTFVPQNQTIESPDNALQFRLYGKDYNVYTHSFLCYGKDQALWQKL
AKDIQVASNEILRDPCFHPGYKKVVNVSDLYKTPCTKRFEMTLPFQQFE
IQGIGNYQQCHQSILELFNTSYCPYSQCAFNGIFLPPLQGDFGAFSAFY
FVMKFLNLTSEKVSQEKVTEMMKKFCAQPWEEIKTSYAGVKEKYLSEYC
FSGTYILSLLLQGYHFTADSWEHIHFIGKIQGSDAGWTLGYMLNLTNMI
PAEQPLSTPLSHSTTRGGPEQKLISEEDLNSAVDHHHHHH

where:
Uppercase italic text corresponds to a leader sequence [SEQ ID NO:30] used for expression of the construct;
Uppercase regular text corresponds to the anti-GPIIb/IIIa (CD41) scFv;
Uppercase bold text corresponds to the human serum albumin sequence
Uppercase underlined text corresponds to the amino acid sequence of the extracellular domain of CD39; and
A His tag and a C-myc tag are underlined and italicized.

Other scFv-CD39 fusion proteins comprising human serum albumin that are produced using the same methods are shown below:

SEQ ID NO: 33
EVQLVESGGGLVQPGGSLRLSCAASGFMFSRYAMSWVRQAPGKGPEWVS
GISGSGGSTYYADSVKGRFTVSRDNSKNTLYLQMNSLRAEDTAVYYCAR
IFTHRSRGDVPDQTSFDYWGQGTLVTVSSGSASAPKLEEGEFSEARVSS
ELTQDPAVSVALGQTVRITCQGDSLRNFYASWYQQKPGQAPTLVIYGLS
KRPSGIPDRFSASSSGNTASLTITGAQAEDEADYYCLLYYGGGQQGVFG
GGTKLTVL [symbol] AHKSEVAHRFKDLGEENFKALVLIAFAQYLQQCPFEDHV
KLVNEVTEFAKTCVADESAENCDKSLHTLFGDKLCTVATLRETYGEMAD
CCAKQEPERNECFLQHKDDNPNLPRLVRPEVDVMCTAFHDNEETFLKKY
LYEIARRHPYFYAPELLFFAKRYKAAFTECCQAADKAACLLPKLDELRD
EGKASSAKQRLKCASLQKFGERAFKAWAVARLSQRFPKAEFAEVSKLVT
DLTKVHTECCHGDLLECADDRADLAKYICENQDSISSKLKECCEKPLLE
KSHCIAEVENDEMPADLPSLAADFVESKDVCKNYAEAKDVFLGMFLYEY
ARRHPDYSVVLLLRLAKTYETTLEKCCAAADPHECYAKVFDEFKPLVEE
PQNLIKQNCELFEQLGEYKFQNALLVRYTKKVPQVSTPTLVEVSRNLGK
VGSKCCKHPEAKRMPCAEDYLSVVLNQLCVLHEKTPVSDRVTKCCTESL
VNRRPCFSALEVDETYVPKEFNAETFTFHADICTLSEKERQIKKQTALV
ELVKHKPKATKEQLKAVMDDFAAFVEKCCKADDKETCFAEEGKKLVAAS
QAALGLGG [symbol] TQNKALPENVKYGIVLDAGSSHTSLYIYKWPAEKENDTG
VVHQVEECRVKGPGISKFVQKVNEIGIYLTDCMERAREVIPRSQHQETP
VYLGATAGMRLLRMESEELADRVLDVVERSLSNYPFDFQGARIITGQEE
GAYGWITINYLLGKFSQKTRWFSIVPYETNNQETFGALDLGGASTQVTF
VPQNQTIESPDNALQFRLYGKDYNVYTHSFLCYGKDQALWQKLAKDIQV
ASNEILRDPCFHPGYKKVVNVSDLYKTPCTKRFEMTLPFQQFEIQGIGN
YQQCHQSILELFNTSYCPYSQCAFNGIFLPPLQGDFGAFSAFYFVMKFL
NLTSEKVSQEKVTEMMKKFCAQPWEEIKTSYAGVKEKYLSEYCFSGTYI
LSLLLQGYHFTADSWEHIHFIGKIQGSDAGWTLGYMLNLTNMIPAEQPL
STPLSHST

where:
Uppercase regular text corresponds to the anti-GPIIb/IIIa (CD41) scFv;
X₁ is an optional linker that is suitably a flexible linker (e.g., [GGGGS]n, wherein n is an integer from 1 to 10, suitably 1 to 5, more suitably 1 to 3);
Uppercase bold text corresponds to the human serum albumin sequence
X₂ is an optional linker that is suitably a flexible linker (e.g., [GGGGS]n, wherein n is an integer from 1 to 10, suitably 1 to 5, more suitably 1 to 3); and
Uppercase underlined text corresponds to the amino acid sequence of the extracellular domain of CD39.

SEQ ID NO: 34
METDTLLLWVLLLWVPGSTGDAAQPARRAMAEVQLVESGGGLVQPGGSL
RLSCAASGFMFSRYAMSWVRQAPGKGPEWVSGISGSGGSTYYADSVKGR
FTVSRDNSKNTLYLQMNSLRAEDTAVYYCARIFTHRSRGDVPDQTSFDY
WGQGTLVTVSSGSASAPKLEEGEFSEARVSSELTQDPAVSVALGQTVRI
TCQGDSLRNFYASWYQQKPGQAPTLVIYGLSKRPSGIPDRFSASSSGNT
ASLTITGAQAEDEADYYCLLYYGGGQQGVFGGGTKLTVLGGGGSAHKSE
VAHRFKDLGEENFKALVLIAFAQYLQQCPFEDHVKLVNEVTEFAKTCVA
DESAENCDKSLHTLFGDKLCTVATLRETYGEMADCCAKQEPERNECFLQ
HKDDNPNLPRLVRPEVDVMCTAFHDNEETFLKKYLYEIARRHPYFYAPE
LLFFAKRYKAAFTECCQAADKAACLLPKLDELRDEGKASSAKQRLKCAS
LQKFGERAFKAWAVARLSQRFPKAEFAEVSKLVTDLTKVHTECCHGDLL
ECADDRADLAKYICENQDSISSKLKECCEKPLLEKSHCIAEVENDEMPA
DLPSLAADFVESKDVCKNYAEAKDVFLGMFLYEYARRHPDYSVVLLLRL
AKTYETTLEKCCAAADPHECYAKVFDEFKPLVEEPQNLIKQNCELFEQL
GEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDET
YVPKEFNAETFTFHADICTLSEKERQIKKQTALVELVKHKPKATKEQLK
AVMDDFAAFVEKCCKADDKETCFAEEGKKLVAASQAALGLGGGGGGSTQ
NKALPENVKYGIVLDAGSSHTSLYIYKWPAEKENDTGVVHQVEECRVKG
PGISKFVQKVNEIGIYLTDCMERAREVIPRSQHQETPVYLGATAGMRLL
RMESEELADRVLDVVERSLSNYPFDFQGARIITGQEEGAYGWITINYLL
GKFSQKTRWFSIVPYETNNQETFGALDLGGASTQVTFVPQNQTIESPDN
ALQFRLYGKDYNVYTHSFLCYGKDQALWQKLAKDIQVASNEILRDPCFH
PGYKKVVNVSDLYKTPCTKRFEMTLPFQQFEIQGIGNYQQCHQSILELF
NTSYCPYSQCAFNGIFLPPLQGDFGAFSAFYFVMKFLNLTSEKVSQEKV
TEMMKKFCAQPWEEIKTSYAGVKEKYLSEYCFSGTYILSLLLQGYHFTA
DSWEHIHFIGKIQGSDAGWTLGYMLNLTNMIPAEQPLSTPLSHST

where:
Uppercase italic text corresponds to a leader sequence [SEQ ID NO:30] used for expression of the construct;
Uppercase regular text corresponds to the anti-GPIIb/IIIa (CD41) scFv;
Uppercase bold text corresponds to the human serum albumin sequence; and
Uppercase underlined text corresponds to the amino acid sequence of the extracellular domain of CD39.

SEQ ID NO: 35
METDTLLLWVLLLWVPGSTGDAAQPARRAMAEVQLVESGGGLVQPGGSL
RLSCAASGFMFSRYAMSWVRQAPGKGPEWVSGISGSGGSTYYADSVKGR
FTVSRDNSKNTLYLQMNSLRAEDTAVYYCARIFTHRSRGDVPDQTSFDY
WGQGTLVTVSSGSASAPKLEEGEFSEARVSSELTQDPAVSVALGQTVRI
TCQGDSLRNFYASWYQQKPGQAPTLVIYGLSKRPSGIPDRFSASSSGNT
ASLTITGAQAEDEADYYCLLYYGGGQQGVFGGGTKLTVLGGGGSAHKSE
VAHRFKDLGEENFKALVLIAFAQYLQQCPFEDHVKLVNEVTEFAKTCVA
DESAENCDKSLHTLFGDKLCTVATLRETYGEMADCCAKQEPERNECFLQ
HKDDNPNLPRLVRPEVDVMCTAFHDNEETFLKKYLYEIARRHPYFYAPE
LLFFAKRYKAAFTECCQAADKAACLLPKLDELRDEGKASSAKQRLKCAS
LQKFGERAFKAWAVARLSQRFPKAEFAEVSKLVTDLTKVHTECCHGDLL
ECADDRADLAKYICENQDSISSKLKECCEKPLLEKSHCIAEVENDEMPA
DLPSLAADFVESKDVCKNYAEAKDVFLGMFLYEYARRHPDYSVVLLLRL
AKTYETTLEKCCAAADPHECYAKVFDEFKPLVEEPQNLIKQNCELFEQL
GEYKFQNALLVRYTKKVPQVSTPTLVEVSRNLGKVGSKCCKHPEAKRMP
CAEDYLSVVLNQLCVLHEKTPVSDRVTKCCTESLVNRRPCFSALEVDET
YVPKEFNAETFTFHADICTLSEKERQIKKQTALVELVKHKPKATKEQLK
AVMDDFAAFVEKCCKADDKETCFAEEGKKLVAASQAALGLGGGGGGSTQ
NKALPENVKYGIVLDAGSSHTSLYIYKWPAEKENDTGVVHQVEECRVKG
PGISKFVQKVNEIGIYLTDCMERAREVIPRSQHQETPVYLGATAGMRLL
RMESEELADRVLDVVERSLSNYPFDFQGARIITGQEEGAYGWITINYLL
GKFSQKTRWFSIVPYETNNQETFGALDLGGASTQVTFVPQNQTIESPDN
ALQFRLYGKDYNVYTHSFLCYGKDQALWQKLAKDIQVASNEILRDPCFH
PGYKKVVNVSDLYKTPCTKRFEMTLPFQQFEIQGIGNYQQCHQSILELF
NTSYCPYSQCAFNGIFLPPLQGDFGAFSAFYFVMKFLNLTSEKVSQEKV
TEMMKKFCAQPWEEIKTSYAGVKEKYLSEYCFSGTYILSLLLQGYHFTA
DSWEHIHFIGKIQGSDAGWTLGYMLNLTNMIPAEQPLSTPLSHSTTRGG
PEQKLISEEDLNSAVDHHHHHH

where:
Uppercase italic text corresponds to a leader sequence [SEQ ID NO:30] used for expression of the construct;
Uppercase regular text corresponds to the anti-GPIIb/IIIa (CD41) scFv;
Uppercase bold text corresponds to the human serum albumin sequence;
Uppercase underlined text corresponds to the amino acid sequence of the extracellular domain of CD39; and
His tags and a C-myc tag are underlined and italicized.

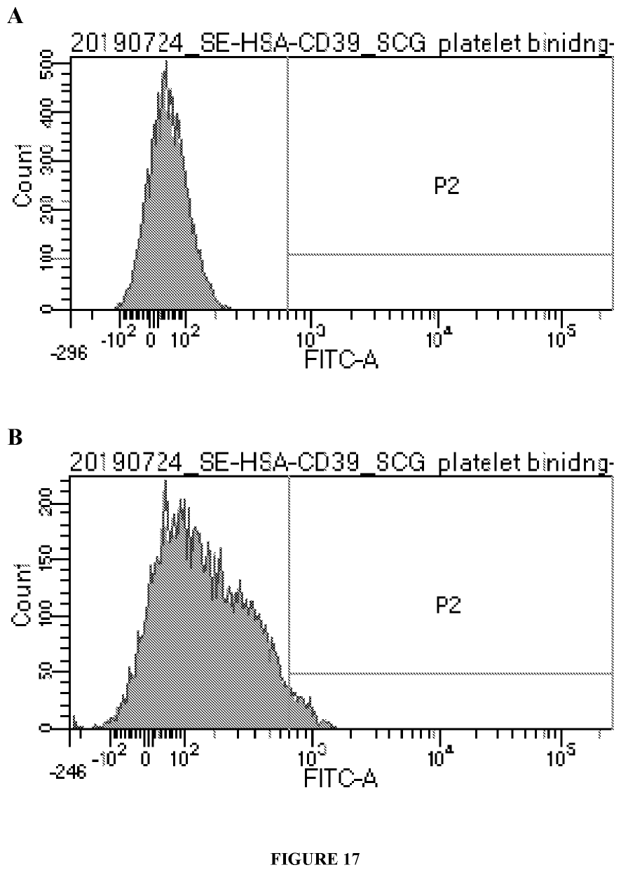

The scFv-HSA-CD39 construct was tested for its ability to bind to human platelets using the methods described in Example 14. FIG. 17 shows that, like scFv-CD39, the scFv-HSA-CD39 construct binds to activated human platelets but not non-activated human platelets.

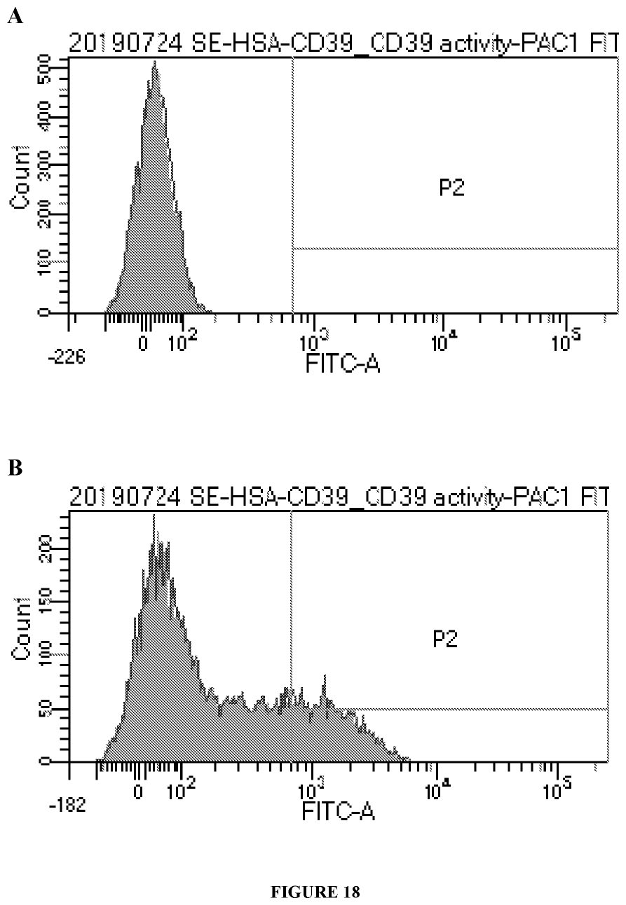

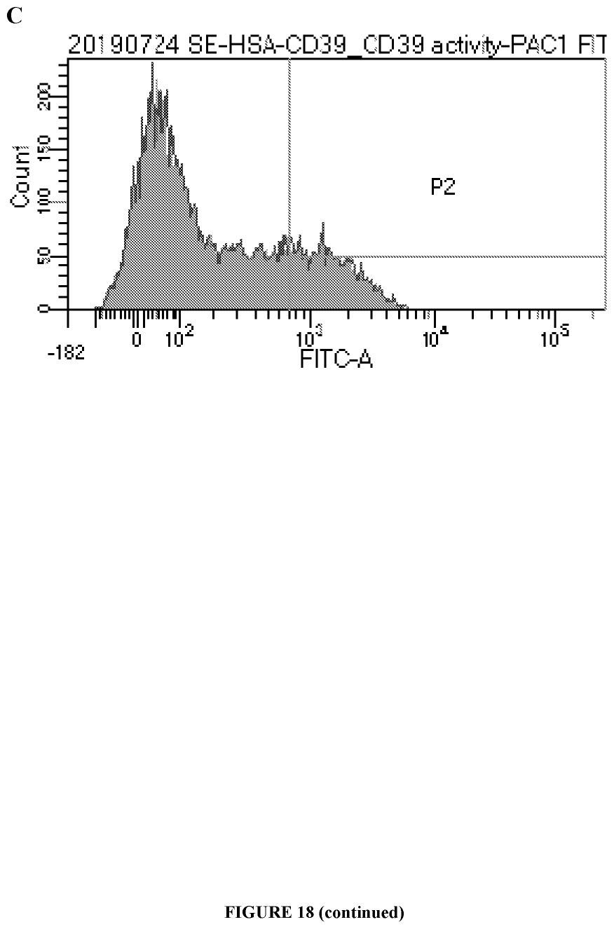

Using similar methods, the scFv-HSA-CD39 construct was also tested for its ability to hydrolyse ADP to confirm that the CD39 portion was active. FIG. 18 shows that scFv-HSA-CD39 inhibited binding of PAC-1 FITC (a fluorescently labelled antibody that also binds to GPIIb/IIIa on activated platelets) to platelets by hydrolysing 20 nM ADP (FIG. 18C), thereby preventing activation of the platelets to permit PAC-1 FITC binding. PAC-1 FITC did not bind to non-activated platelets (no ADP; FIG. 18A), but was able to bind to platelets that were activated with 20 nM ADP in the absence of scFv-HSA-CD39 (FIG. 18B).

These results confirm that scFv-CD39 fusion proteins further comprising a HSA sequence retain their ability to target activated platelets and their CD39 activity.

The present application claims priority from AU 2019901808, filed on 27 May 2019, the entire contents of which are incorporated herein by reference.

All publications cited herein are hereby incorporated by reference in their entirety. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A binding protein comprising an extracellular domain of CD39 and a binding region that specifically binds to activated glycoprotein (GP)IIb/IIIa.

2. The binding protein of claim 1, wherein the extracellular domain of CD39 comprises or consists of a sequence set forth in SEQ ID NO: 4.

3. The binding protein of claim 1 or 2, wherein the binding region specifically binds an epitope on GPIIb/IIIa recognised by a scFV consisting of a sequence set forth in SEQ ID NO: 1.

4. The binding protein of any one of claims 1 to 3, wherein the binding region comprises an antibody variable region that binds to or specifically binds to GPIIb/IIIa and neutralizes GPIIb/IIIa receptor function and/or activity.

5. The binding protein of any one of claims 1 to 4, wherein the binding region is a protein comprising a Fv.

6. The binding protein of claim 5, wherein the protein comprises a single chain Fv fragment (scFv).

7. The binding protein of any one of claims 1 to 6, wherein the binding protein is a fusion protein.

8. The binding protein of any one of claims 1 to 7, wherein the binding protein comprises a sequence which is at least 90% identical to a sequence set forth in SEQ ID NO: 1.

9. The binding protein of any one of claims 1 to 7, wherein the binding protein comprises a sequence which is at least 90% identical to a sequence set forth in SEQ ID NO: 23

10. The binding protein of any one of claims 1 to 9, wherein the binding protein comprises a heavy chain variable region (V_{H}) comprising a sequence which is at least 90% identical to a sequence set forth in SEQ ID NO: 2 and a light chain variable region (V_{L}) comprising a sequence which is at least 90% identical to a sequence set forth in SEQ ID NO: 3.

11. The binding protein of any one of claims 1 to 9, wherein the binding protein comprises a heavy chain variable region (V_{H}) comprising a sequence which is at least 90% identical to a sequence set forth in SEQ ID NO: 21 and a light chain variable region (V_{L}) comprising a sequence which is at least 90% identical to a sequence set forth in SEQ ID NO: 22.

12. The binding protein of any one of claims 1 to 11, wherein the binding protein comprises complementarity determining regions (CDRs) of the V_{H} of SEQ ID NO: 2 and/or the CDRs of the V_{L} of SEQ ID NO: 3.

13. The binding protein of any one of claims 1 to 12, wherein the extracellular domain of CD39 is linked to the binding region via a linker.

14. The binding protein of claim 13, wherein the linker is a peptide linker comprising between 3 and 30 amino acids in length.

15. The binding protein of any one of claims 1 to 14, wherein the binding protein comprises a sequence set forth in SEQ ID NO: 6.

16. The binding protein of any one of claims 1 to 14, further comprising a human serum albumin.

17. The binding protein of claim 16, wherein the human serum albumin comprises a sequence which is at least 90% identical to a sequence set forth in SEQ ID NO: 31.

18. A composition comprising the binding protein of any one of claims 1 to 17 and a pharmaceutically acceptable carrier.

19. A method of treating or preventing an inflammatory neurological disease in a subject, the method comprising administering to the subject the binding protein of any one claims 1 to 17 or the composition of claim 18.

20. A method of treating or preventing an inflammatory neurological disease in a subject, the method comprising administering to the subject a protein comprising an extracellular domain of CD39.

21. The method of claim 20, wherein the protein is a binding protein.

22. The method of claim 21, wherein the binding protein comprises a binding region that specifically binds to activated glycoprotein (GP)IIb/IIIa.

23. The method of any one of claims 20 to 22, wherein the inflammatory neurological disease is a degenerative disease of the central nervous system.

24. The method of claim 23, wherein the degenerative disease of the central nervous system is multiple sclerosis.

25. The method of any one of claims 20 to 24, wherein the extracellular domain of CD39 comprises or consists of a sequence set forth in SEQ ID NO: 4.

26. The method of any one of claims 22 to 25, wherein the binding region specifically binds an epitope on GPIIb/IIIa recognised by a scFV consisting of a sequence set forth in SEQ ID NO: 1.

27. The method of any one of claims 22 to 26, wherein the binding region comprises an antibody variable region that binds to or specifically binds to GPIIb/IIIa and neutralizes GPIIb/IIIa receptor function and/or activity.

28. The method of any one of claims 12 to 27, wherein the binding region is a protein comprising a Fv.

29. The method of claim 28, wherein the protein comprises a single chain Fv fragment (scFv).

30. The method of any one of claims 22 to 29, wherein the binding protein is a fusion protein.

31. The method of any one of claims 22 to 30, wherein the binding protein comprises a sequence which is at least 90% identical to a sequence set forth in SEQ ID NO: 1.

32. The method of any one of claims 22 to 30, wherein the binding protein comprises a sequence which is at least 90% identical to a sequence set forth in SEQ ID NO: 23.

33. The method of any one of claims 22 to 32, wherein the binding protein comprises a heavy chain variable region (V_{H}) comprising a sequence which is at least 90% identical to a sequence set forth in SEQ ID NO: 2 and a light chain variable region (V_{L}) comprising a sequence which is at least 90% identical to a sequence set forth in SEQ ID NO: 3.

34. The method of any one of claims 22 to 32, wherein the binding protein comprises a heavy chain variable region (V_{H}) comprising a sequence which is at least 90% identical to a sequence set forth in SEQ ID NO: 21 and a light chain variable region (V_{L}) comprising a sequence which is at least 90% identical to a sequence set forth in SEQ ID NO: 22.

35. The method of any one of claims 22 to 34, wherein the binding protein comprises the complementarity determining regions (CDRs) of the V_{H} of SEQ ID NO: 2 and/or the CDRs of the V_{L} of SEQ ID NO: 3.

36. The method of any one of claims 22 to 35, wherein the extracellular domain of CD39 is linked to the binding region via a linker.

37. The method of claim 36, wherein the linker is a peptide linker comprising between 3 and 30 amino acids in length.

38. The method of any one of claims 22 to 37, wherein the binding protein comprises a sequence set forth in SEQ ID NO: 6.

39. The method of any one of claims 22 to 37, wherein the binding protein further comprises a human serum albumin.

40. The method of claim 39, wherein the human serum albumin comprises a sequence which is at least 90% identical to a sequence set forth in SEQ ID NO: 31.

41. The method of any one of claims 20 to 40, wherein the subject is at risk of developing multiple sclerosis or a symptom thereof.

42. The method of any one of claims 22 to 41, wherein the binding protein is administered in an amount effective to:
decrease plasma levels of adenosine-5′-diphosphate (ADP);
reduce and/or prevent platelet accumulation and/or platelet infiltration in the CNS parenchyma;
reduce and/or prevent astrocytic and/or microglial glial reactivity;
reduce and/or prevent demyelination; and/or
reduce and/or prevent lymphocytic infiltration.

43. The method of any one of claims 22 to 42, wherein the binding protein is administered prior to the onset of clinical symptom(s) of the inflammatory neurological disease.

44. The method of claim 43, wherein the onset of clinical symptom(s) is characterised by an increase in circulating platelet numbers.

45. Use of a protein in the manufacture of a medicament for treating or preventing an inflammatory neurological disease in a subject, wherein the protein comprises an extracellular domain of CD39.

46. Use of a binding protein in the manufacture of a medicament for treating or preventing an inflammatory neurological disease in a subject, wherein the binding protein comprises:
an extracellular domain of CD39; and
a binding region that specifically binds to activated glycoprotein (GP)IIb/IIIa.

47. A kit for use in the treatment or prevention of an inflammatory neurological disease in a subject, the kit comprising:
(i) at least one protein comprising an extracellular domain of CD39;
(ii) instructions for using the kit in treating or preventing the inflammatory neurological disease in the subject; and
(iii) optionally, at least one further therapeutically active compound or drug.

48. A kit for use in the treatment or prevention of an inflammatory neurological disease in a subject, the kit comprising:
(i) at least one binding protein comprising
a. an extracellular domain of CD39; and
b. binding region that specifically binds to activated glycoprotein (GP)IIb/IIIa;
(ii) instructions for using the kit in treating or preventing the inflammatory neurological disease in the subject; and
(iii) optionally, at least one further therapeutically active compound or drug.
